(19) United States
(12) Patent Application Publication
(10) Pub. No.: US 2017/0290516 A1
(43) Pub. Date: Oct. 12, 2017

(54) Determination of Physiological Age

(71) Applicant: Segterra, Inc., Cambridge, MA (US)

(72) Inventors: Raymond Nguyen, Auburndale, MA (US); Gil Blander, Lexington, MA (US); Rony Sellam, Brookline, MA (US); Mark Allan McCormick, San Francisco, CA (US); David Andrew Sinclair, Chestnut Hill, MA (US); Leonard P. Guarente, Auburndale, MA (US)

(21) Appl. No.: 15/507,839

(22) PCT Filed: Sep. 1, 2015
(86) PCT No.: PCT/US2015/047945

§ 371 Date: Mar. 1, 2017

Related U.S. Application Data
(60) Provisional application No. 62/044,546, filed on Sep. 2, 2014.

(51) Int. Cl. A61B 5/02; A61B 5/0245

(52) U.S. Cl. CPC A61B 5/02; A61B 5/0245; A61B 5/00

(57) ABSTRACT

The technology described in this document can be embodied in a computer-implemented method that includes accessing, by one or more processors, data for a plurality of health-related parameters of an individual, and determining, by the one or more processors, a corresponding individual age-adjustment factor based on data for one or more of the plurality of health-related parameters. The age-adjustment factor computed for a given health-related parameter represents an estimated gain or loss in age as indicated by the data for the given health-related parameter. The method also includes combining, by the one or more processors, two or more of the individual age-adjustment factors to provide a combined age-adjustment factor, and determining, by the one or more processors, a physiological age of the individual based on the combined age-adjustment factor and a chronological age of the individual.

### PRIORITY CLAIM

This application is a 371 U.S. National of PCT/US2015/047945, filed on Sep. 1, 2015, which claims priority to U.S. Provisional Application No. 62/044,546, filed on Sep. 2, 2014, the entire content of which is incorporated herein by reference.

### TECHNICAL FIELD

This specification describes technologies related to evaluating health and fitness parameters.

### BACKGROUND

Various biomarkers and physiological markers from the human body can be used in evaluating health and fitness of individuals.

### SUMMARY

In one aspect, the present disclosure features a computer-implemented method that includes accessing, by one or more processors, data for a plurality of health-related parameters of an individual, and determining, by the one or more processors, a corresponding individual age-adjustment factor based on data for one or more of the plurality of health-related parameters. The age-adjustment factor computed for a given health-related parameter represents an estimated gain or loss in age as indicated by the data for the given health-related parameter. The method also includes combining, by the one or more processors, two or more of the individual age-adjustment factors to provide a combined age-adjustment factor, and determining, by the one or more processors, a physiological age of the individual based on the combined age-adjustment factor and a chronological age of the individual.

In another aspect, the disclosure features a system that includes a memory device and an age analysis engine including a processor. The age analysis engine is configured to access data for a plurality of health-related parameters of an individual, and determine a corresponding individual age-adjustment factor based on data for one or more of the plurality of health-related parameters. The age-adjustment factor computed for a given health-related parameter represents an estimated gain or loss in age as indicated by the data for the given health-related parameter. The age analysis engine is further configured to combine two or more of the individual age-adjustment factors to provide a combined age-adjustment factor, and determine a physiological age of the individual based on the combined age-adjustment factor and a chronological age of the individual.

In another aspect, the disclosure features a non-transitory computer readable storage device having encoded thereon computer readable instructions, which when executed, cause one or more processors to perform various operations. The operations include accessing data for a plurality of health-related parameters of an individual, and determining, a corresponding individual age-adjustment factor based on data for one or more of the plurality of health-related parameters. The age-adjustment factor computed for a given health-related parameter represents an estimated gain or loss in age as indicated by the data for the given health-related parameter. The operations also include combining, two or more of the individual age-adjustment factors to provide a combined age-adjustment factor, and determining, a physiological age of the individual based on the combined age-adjustment factor and a chronological age of the individual.

The data for the plurality of health-related parameters can include one or more of: data on levels of one or more biomarkers, data on levels of one or more physiological markers, and data on one or more lifestyle-related parameters. The biomarkers can include one or more of glucose, testosterone, vitamin D, C-reactive protein (CRP), and alanine aminotransferase (ALT). The physiological markers can include heart rate variability (HRV), pulse pressure, and body mass index (BMI). The data on the one or more lifestyle-related parameters can include one or more of: data on smoking habits and data on activity level. Determining an individual age-adjustment factor for a particular health related parameter can include determining an estimated age of the individual based on the data for the particular health-related parameter, and determining the individual age adjustment factor as a difference between the chronological age and the estimated age. The estimated age can be determined using an equation that relates the particular health parameter to the estimated age. Determining an individual age-adjustment factor for a particular health related parameter can include estimating a projected lifespan for the individual based on the chronological age of the individual and the data for the particular health-related parameter, determining a standard expected lifespan for the individual based on accessing life-expectancy data for a population of which the individual is a part, and determining the individual age adjustment factor as a difference between the projected lifespan and the standard expected lifespan. The projected lifespan can be estimated by accessing a look-up table that stores projected lifespan values as a function of tuples comprising the chronological age of the individual and the data for the particular health-related parameter. The standard expected lifespan can be determined by accessing life-expectancy data in an actuarial life table. The individual age-adjustment factor determined for a health-related parameter can be capped or scaled such that the individual age-adjustment factor is within a predetermined range. Determining the age-adjustment factor can include receiving user-input in response to one or more questions presented to the individual, and determining the individual age adjustment factor by accessing a look-up table based on the user-input. The health related parameters can be obtained using a wearable device worn by the individual. The health related parameters can include genetic markers.

In another aspect, the disclosure features a computer-implemented method for providing a dietary recommendation for an individual. The method includes accessing, by one or more processors, data for a plurality of health-related parameters of an individual, and identifying, by the one or more processors, a set of one or more out-of-range parameters from the plurality of health-related parameters. The method also includes identifying one or more food items from a database of food items, such that the identified food items are related to improving the corresponding levels of the one or more out-of-range parameters, and displaying the identified food items as the dietary recommendation on a user interface presented on a display device.

In another aspect, the disclosure features a system that includes a memory device and a recommendation engine that includes one or more processors. The recommendation engine is configured to access data for a plurality of health-related parameters of an individual, and identify a set of one or more out-of-range parameters from the plurality of health-related parameters. The recommendation engine is further configured to identify one or more food items from a database of food items, such that the identified food items are related to improving the corresponding levels of the one or more out-of-range parameters, and display the identified food items as the dietary recommendation on a user interface presented on a display device.

In another aspect, the disclosure features a non-transitory computer readable storage device having encoded thereon computer readable instructions, which when executed, cause one or more processors to perform various operations. The operations include accessing data for a plurality of health-related parameters of an individual, and identifying, a set of one or more out-of-range parameters from the plurality of health-related parameters. The operations also include identifying one or more food items from a database of food items, such that the identified food items are related to improving the corresponding levels of the one or more out-of-range parameters, and displaying the identified food items as the dietary recommendation on a user interface presented on a display device.

The identified food items can include one or more food items to consume. The identified food items can include one or food items to avoid. The database of food items can include information on macronutrients and micronutrients associated with the food items. The one or more food items can be identified such that the identified food items do not adversely affect a level of a health-related parameter not included in the set of one or more out-of-range parameters. Identifying the one or more food items can include identifying one or more nutrients known to affect the set of one or more out-of-range parameters, and comparing a set of candidate food items based on corresponding normalized amounts of the one or more nutrients in the candidate food items. The normalization can be done with respect to predetermined serving sizes corresponding to the candidate food items. The one or more food items can be selected from the candidate food items based on the comparison. The one or more food items can be identified based also on one or more of (i) availability information for the one or more food items, (ii) price of the one or more food items, and (iii) dietary preferences of the individual. Identifying the one or more food items can include identifying at least one food item from each of a predetermined number of food groups.

The details of one or more implementations of the subject matter described in this specification are set forth in the accompanying drawings and the description below. Other potential features, aspects, and advantages of the subject matter will become apparent from the description, the drawings, and the claims.

### BRIEF DESCRIPTION OF THE DRAWINGS

FIG. 1 is a block diagram of a system used in determining physiological age of an individual.

FIG. 2A is a flowchart for an example process used in calculating an age adjustment factor for the biomarker glucose.

FIGS. 2B-2I are various examples of data used in calculating age adjustment factors using the process of FIG. 2A.

FIG. 3A is a flowchart for an example process used in calculating an age adjustment factor for biomarkers and physiological markers.

FIGS. 3B-3H are various examples of data used in calculating age adjustment factors using the process of FIG. 3A.

FIGS. 4A-4H are examples of user interfaces.

FIG. 5 is a flowchart for an example process for determining a physiological age of an individual.

FIG. 6 is a flowchart for an example process for providing dietary recommendations to an individual.

FIG. 7 is a block diagram of an example of a computing system

### DETAILED DESCRIPTION

Chronological age can be precisely defined as the amount of time that has passed since the birth of an individual. However, the effect of chronological age on individuals may vary. The aging process may be manifested, for example, as a combination of various changes that affect a person over time. These changes can include, for example, physical and psychological changes, as well as changes in lifestyle and social interactions. From a biological standpoint, aging may be manifested as a progressive decline of physiological integrity that eventually leads to mortality. The rate at which the various changes affect individuals (i.e., the rate of aging) varies from person to person and can be influenced by a variety of factors such as internal factors (e.g., genetics) and external factors (e.g., lifestyle choices). As a result, people with the same chronological age can have substantially different health status. The health and fitness status of an individual can be represented as a “physiological age” of the individual. For example, a person whose chronological age is 35 may have health/fitness that is typical of a 45 year old healthy person. In such a case, the physiological age of the person can be represented as 45. On the other hand, if a 35 year old person is healthier and fitter than what is expected for a typical 35 year old person, his/her physiological age may be represented, for example, as 30 years. In such a case, the health/fitness status of the person may be viewed as being equivalent to the health/fitness status of a typical 30 year old person.

The physiological age can be determined, for example, using levels of various biomarkers and/or physiological markers present in the body. Therefore, if the physiological age of a male individual is represented as 30 years, the levels of a set of biomarkers/physiological markers in the person can be equivalent to the corresponding “normal” levels found in a typical healthy 30 year old male substantially similar to the individual. The corresponding “normal” levels can be determined based on various factors including, for example, age, gender, ethnicity, activity level, profession, or genetic makeup. The corresponding “normal” levels can be determined based on various studies and can be stored in a database.

As the average human life expectancy increases, the likelihood of developing aging-related diseases can also increase. Worldwide, many deaths occur as a result of age-related causes. The technology described in this document allows for tracking and/or controlling one or more biomarkers and physiological markers that potentially affect one or more age-related conditions and/or chronic diseases such as cardiovascular diseases, hypertension, some forms of cancer, and some forms of diabetes (e.g., type II diabetes). By tracking and/or controlling such biomarkers and physiological markers, a greater degree of control may be exercised on various chronic diseases and other age-related conditions, to keep older adult populations healthy. This can in turn have various long term advantages. For example, by allowing for a greater control over preventable age-related conditions, the technology described herein can contribute to significant savings in resources used in treating such conditions.

A large percentage of adults fail to meet recommended levels of physical activity and/or partake in consumption of poor diet, for example, by eating more than a required daily amount of saturated fat or eating less than five servings of fruit and vegetables per day. By tracking relevant biomarkers and/or physiological markers, the technology described herein can be used to make focused dietary- and lifestyle-related recommendations (or provide other personalized interventions) such that the levels of the corresponding biomarkers may eventually be optimized to levels appropriate levels. The technology may utilize, for example, population-based evidence (e.g., as available from studies, reports and research from various authorities) to provide a comprehensive and interactive depiction of how lifestyle decisions affect the various biomarkers and physiological markers.

Provided herein are methods for determining the physiological age of an individual that include performing an age analysis (e.g., using an age analysis engine as described herein) using values or levels of various biomarkers, physiological markers, and/or lifestyle parameters to determine a physiological age for the individual. The methods can also include providing the individual with personalized recommendations (e.g., dietary, exercise-related or other lifestyle related recommendations) for optimizing, e.g., reducing, increasing, or otherwise controlling the biomarkers and/or physiological markers, to thereby alter (e.g., reduce) the physiological age of the individual. In preferred embodiments, the individual is a human subject. In some embodiments, the methods are computer-implemented methods.

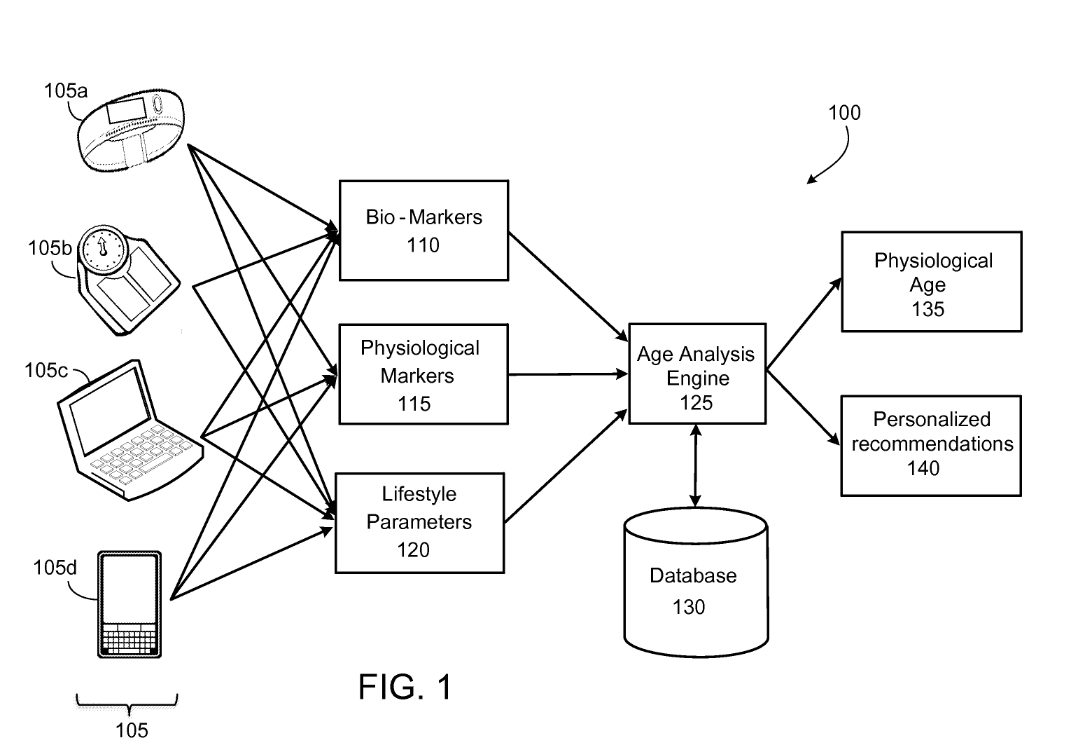

FIG. 1 is a block diagram that shows the example of a system 100 used in determining physiological age of an individual. The system 100 includes an age analysis engine 125 that analyzes various biomarkers 110, physiological markers 115, and/or lifestyle parameters 120 of an individual to determine a physiological age 135 for the individual. The age analysis engine may also provide personalized recommendations 140 (e.g., dietary, exercise-related or other lifestyle related recommendations) for optimizing, reducing, increasing, or otherwise controlling the biomarkers 110 and/or physiological markers 115, to alter (e.g., reduce) the physiological age of the individual.

Information about the various biomarkers 110, physiological markers 115, and/or lifestyle parameters 120 can be obtained, for example, using one or more devices 105. The devices 105 can include, for example, wearable devices 105a such as smart watches or activity trackers that measures, computes, or otherwise provides information on various health/fitness related parameters such as heart rate, calorie consumption, calorie expenditure, distance walked, steps taken, electro cardiogram (ECG), or quality of sleep. The devices 105 can also include non-wearable devices 105b such as weight scales or other scales configured to provide information on weight, body-mass index (BMI), or water content of the body. The devices 105 also include personal computer devices 105c or mobile computing devices 105d through which a user can provide information on one or more of the biomarkers 110, physiological markers 115 and lifestyle parameters 120. For example, the mobile computing device 105d (e.g., a smartphone or tablet) can include one or more applications for measuring a biomarker 110, physiological marker 115 or lifestyle parameter 120. In some implementations, a user can use the computing devices 105c or 105d to provide user input (e.g., presented to the user via a questionnaire or form, or as a part of an application) related to one or more of the biomarkers 110, physiological markers 115, or lifestyle parameters.

In some implementations, the computing devices 105c or 105d can be used to accept user-input on one or more tests such as blood tests, urine test, sputum test, or other tests for determining levels of one or more biomarkers 110, physiological markers 115, and lifestyle parameters 120. In some implementations, the devices 105 can include a testing apparatus (e.g., a glucose meter) that can transmit test results to the age analysis engine, for example over a wireless network such as a Wi-Fi network. In some implementations, the devices 105 can include a testing apparatus that can provide test results to a computing device 105c or 105d for transmission to the age analysis engine 125.

Various biomarkers 110 can be used by the age analysis engine 125 in determining the physiological age 135 of a person. Examples of such biomarkers 110 include total cholesterol, high density lipoprotein (HDL), low density lipoprotein (LDL), glucose, triglyceride, testosterone, estradiol, prolactin, vitamin D, hemoglobin, calcium, parathyroid hormone (PTH), insulin-like growth factor such as IGF-1, tumor necrosis factor (TNF) such as TNF-alpha, pro-inflammatory cytokine such as IL-6, C-reactive protein (CRP), folic Acid, vitamin B12, alanine aminotransferase (ALT), blood urea nitrogen (BUN), ferritin, sodium, zinc, white blood cells, potassium, creatine kinase (CK) and magnesium. In some implementations, telomere length can be used as a biomarker 110. Such biomarkers 110 can be measured, for example, using a device 105 or obtained, for example, via a laboratory test of a body fluid such as blood, urine, sputum, or semen.

Various physiological markers 115 can also be used by the age analysis engine 125 in determining the physiological age 135. Examples of such physiological markers include, for example, heart rate variability, pulse pressure, heart rate, BMI, blood pressure, weight, and height. The physiological markers 115 can be measured, for example, using a wearable device 105a or a non-wearable device 105b, or obtained, for example, via user-inputs through computing devices 105c or 105d.

In some implementations, one or more lifestyle parameters 120 may also be used by the age analysis engine to determine the physiological age 135. Examples of such lifestyle parameters include, for example, activity level (e.g., amount of exercise done per day), smoker status (e.g., whether or not a smoker, time since quitting, number of cigarettes or other units smoked per day etc.).

In some implementations, one or more of the biomarkers 110, physiological markers 115, and lifestyle parameters 120 can be obtained from a remote data source. For example, information on these markers and parameters can be obtained, for example, from the medical records of the individual based on appropriate permissions from the individual and from a remote storage location (e.g., a cloud storage system) storing such records. In some implementations, the markers and parameters can also be obtained from a database 130 accessible to the age analysis engine 125. For example, an individual may upload information on one or more biomarkers 110, physiological markers 115, and lifestyle parameters 120 to the database 130 using, for example, a user interface displayed on a device 105 (e.g., the computing device 105c or the mobile device 105d). The age analysis engine 125 may access the database 130 to retrieve the information to calculate the physiological age 135 of the individual.

The age analysis engine 125 can be implemented as a combination of software and hardware modules to perform the physiological age computations as described herein. The age analysis engine 125 includes one or more processing devices that can be configured to receive or otherwise access information on one or more of the biomarkers 110, physiological markers 115, and lifestyle parameters 120 to calculate the physiological age 135. In some implementations, the age analysis engine 125 includes a server (or multiple servers) that can be configured to execute processes that calculates the physiological age 135 based on information on one or more of the biomarkers 110, physiological markers 115, and lifestyle parameters 120. In some implementations, the age analysis engine 125 can be configured to execute one or more processes to provide personalized recommendations 140 based on the information on one or more of the biomarkers 110, physiological markers 115, and lifestyle parameters 120. In executing the processes to determine the physiological age 135 or the personalized recommendations, the age analysis engine may access the database 130 or another remote data source to obtain auxiliary information used in the processes.

The physiological age 135 and/or the personalized recommendations 140 can be displayed on a display device or stored on a storage device, wherein the display device and/or the storage device is associated with the age analysis engine 125. In some implementations, the age analysis engine 125 can be configured to provide information on the physiological age 135 and/or the personalized recommendations 140 to a remote computing device over a network that includes a Wi-Fi network or the Internet.

The age analysis engine 125 can be configured to determine a corresponding individual age-adjustment factor for each of the one or more biomarkers 110, physiological markers 115, and lifestyle parameters 120, used in computing the physiological age 135. The age-adjustment factor computed for a given marker or parameter can represent an estimated gain or loss in age as indicated by the data for the given marker or parameter. The age analysis engine 125 can be further configured to combine two or more of the individual age-adjustment factors to provide a combined age-adjustment factor. In some implementations, the physiological age 135 can be determined based on the combined age-adjustment factor and a chronological age of the individual. In some implementations, auxiliary data associated with one or more of the age adjustment factors and the chronological age can be stored as a part of the database 130 stored in a storage device accessible to the age analysis engine 125.

The individual age adjustment factors can be computed by the age analysis engine 125 in various ways. Examples of computing individual age adjustment factors are described next with reference to various biomarkers 110, physiological markers 115, and lifestyle parameters 120. Computing individual age adjustment factors for other markers and parameters, and/or computing the age adjustment factors for the described markers and parameters in other ways are possible and within the scope of this disclosure.

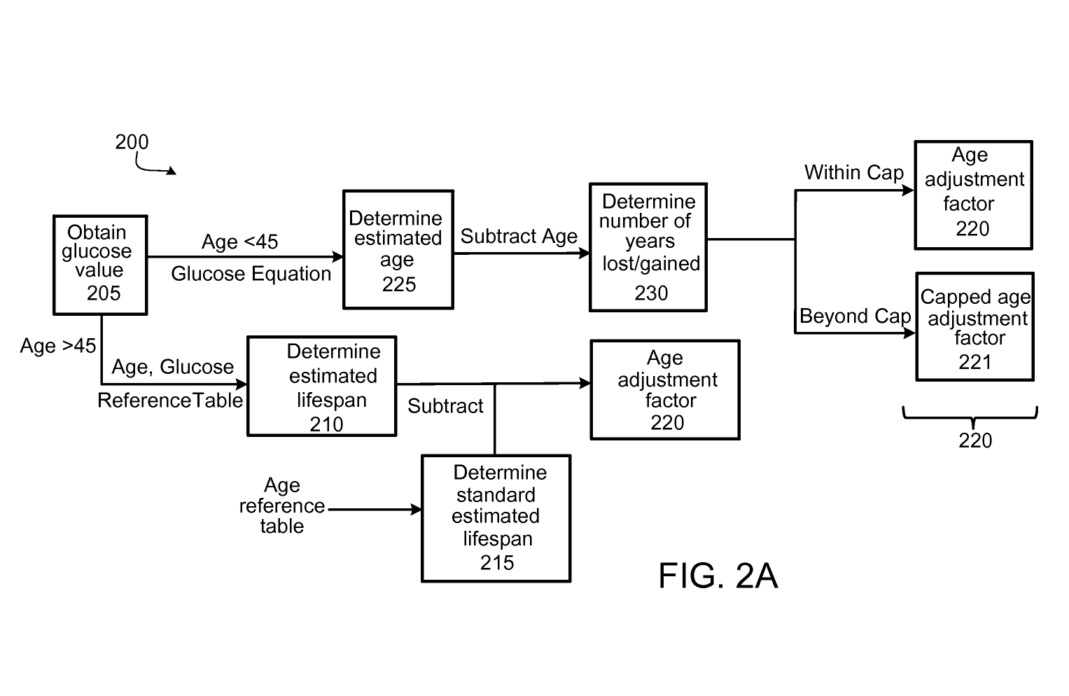

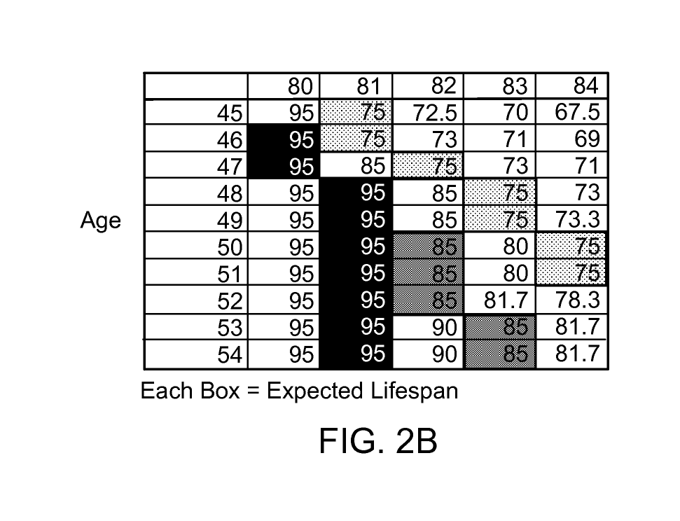

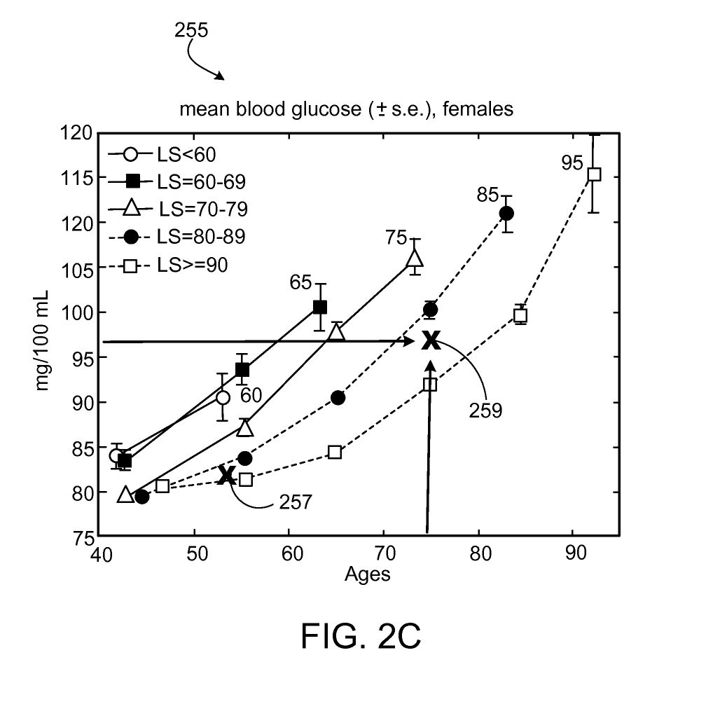

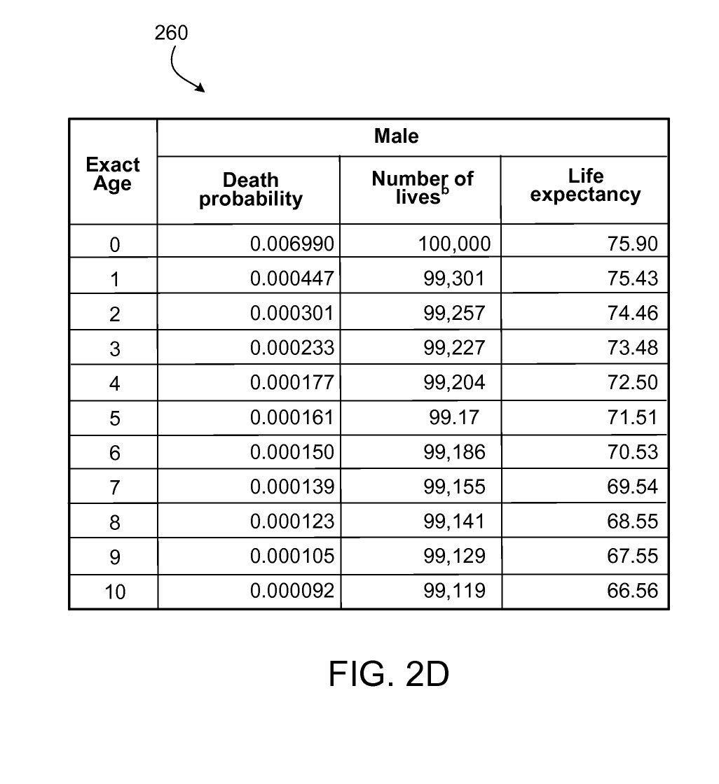

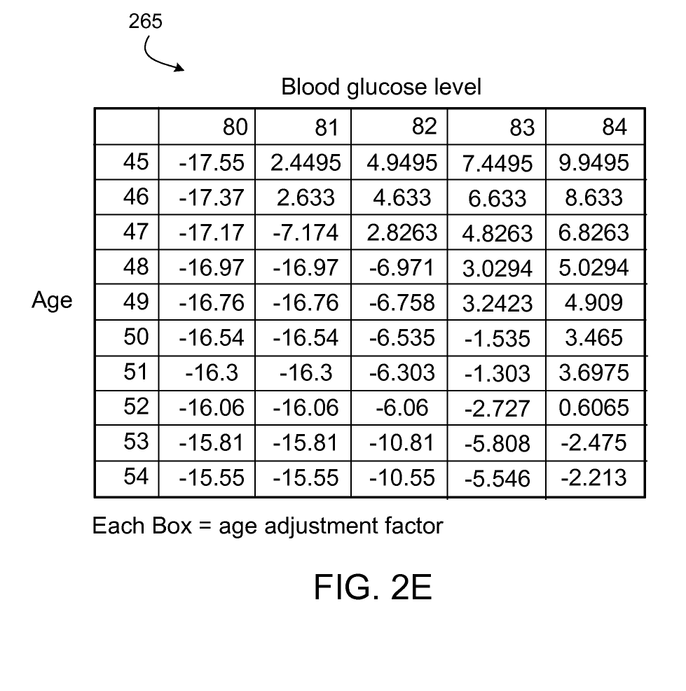

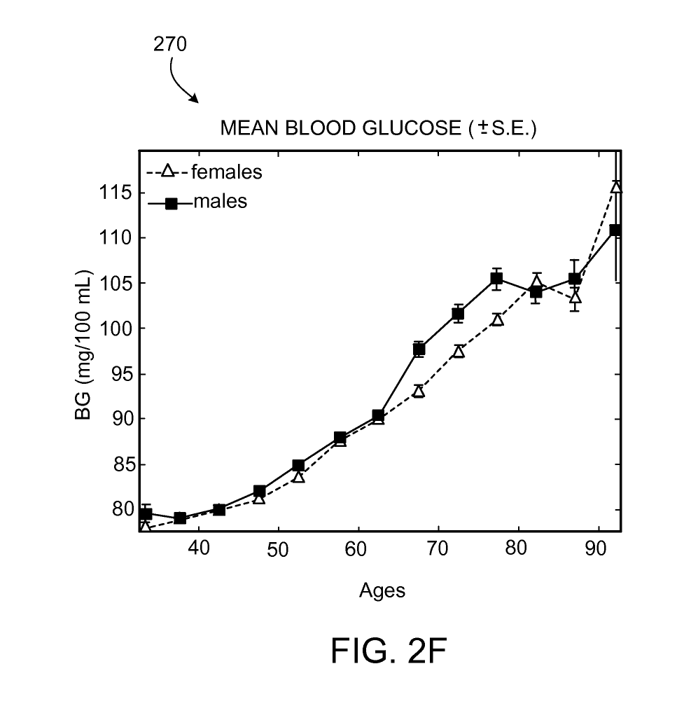

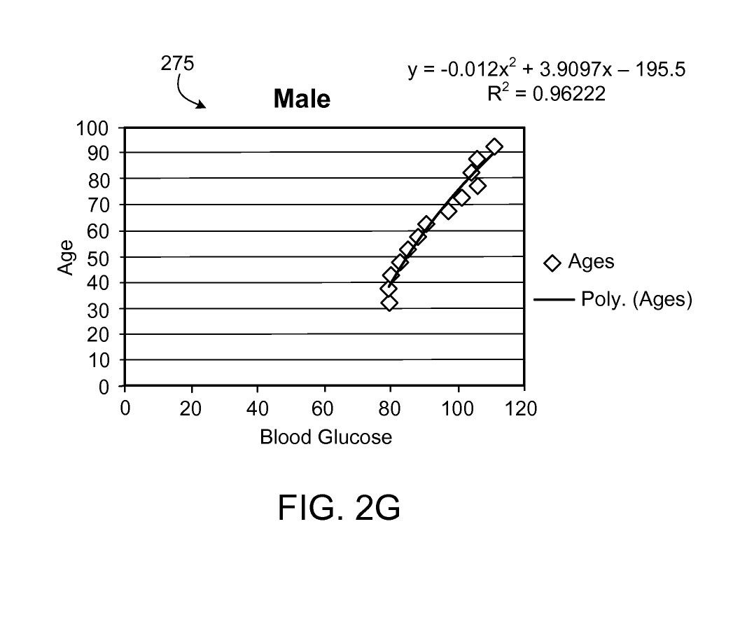

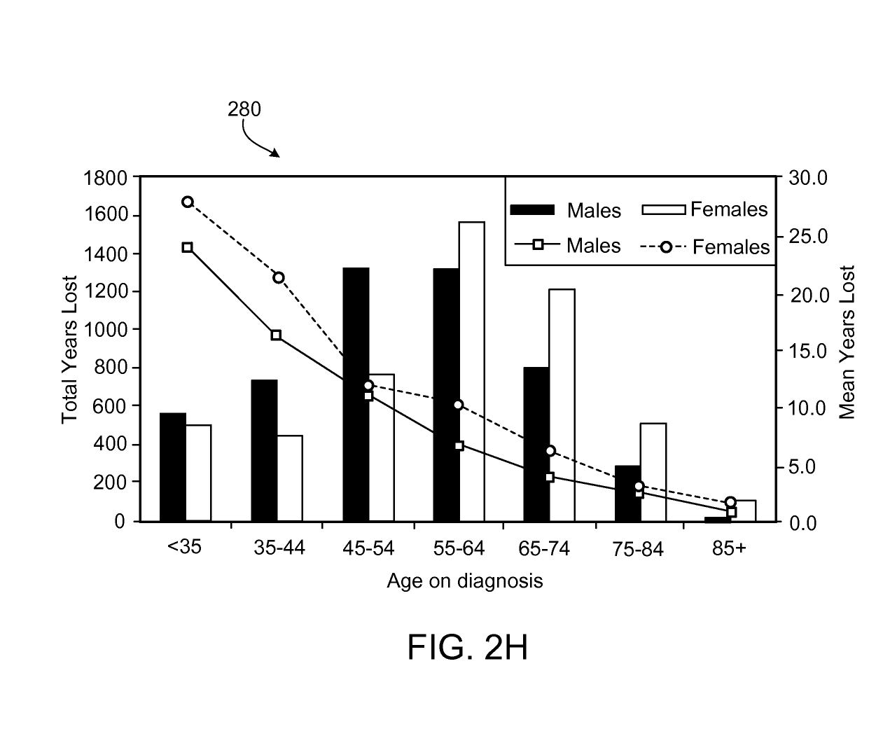

FIG. 2A shows a flowchart for an example process 200 used in calculating an age adjustment factor for the biomarker glucose. The process 200 can be executed by the age analysis engine 125 using one or more processing devices. The operations of the process include obtaining a glucose value for an individual (205). The glucose value can be obtained, for example, using a device 105 or by other ways of obtaining a biomarker 110 as discussed above with reference to FIG. 1. Other information about the individual (e.g., chronological age) is also obtained. In some implementations, the process for calculating the age adjustment factor 220 for glucose can vary based on the age of the individual.

In the example of FIG. 2A, the operations include determining an estimated lifespan for the individual (210) if the age of the individual is more than 45. This can be done, for example, by accessing a reference table that stores estimated lifespans as a function of chronological age and glucose level. An example of such a reference table 250 is shown in FIG. 2B. Each row in the table 250 represents a particular chronological age, and each column represents a blood glucose level. The content of a particular cell in the table 250 represents an expected lifespan for a corresponding combination of chronological age and blood glucose level. For example, if a person of age 49 has a blood glucose level of 80, according to the data in the reference table 250, the expected lifespan of the person would be 95. However, if the blood glucose level for the 49 year old person is 84, the expected lifespan would be 73.3.

The reference table 250 can be created, for example, on research data and studies. In this example, the reference table is created from research data represented in the plot 255 shown in FIG. 2C. The plot 2C is based on research data available in the publication: Yashin, Anatoli I., Ukraintseva, Svetlana V., et al. “Maintaining physiological state for exceptional survival: What is the normal level of blood glucose and does it change with age?” Mechanisms of Ageing and Development. 130 (2009): 611-618. The plot 2C shows data on mean blood glucose level vs. age in females for various lifespans. Each curve represents a particular lifespan and illustrates how the mean blood glucose level changes with age for people having that lifespan.

The reference table 250 can be created, for example, using data represented in the plot 255. If a particular combination of age and blood glucose level can be plotted on or substantially close to a curve, the expected lifespan for the combination is assigned the lifespan associated with the curve. For example, the combination of 54 years and mean blood glucose level of 81 mg/100 ml corresponds to the point 257 on the plot 255. Because the point 257 is sufficiently close to the curve corresponding to a lifespan of 95, the particular combination is assigned an estimated lifespan of 95 years in the reference table 250. Combinations that do not map to a point that is sufficiently close to any particular curve, but falls between two different curves, can be assigned a value based on a vertical distance between the two curves along a line passing through the point. For example, the combination of age 75 years and mean glucose level of about 97 mg/100 ml maps on to the point 259, which lies between the curves for lifespans of 85 years and 95 years. Using an interpolation technique (e.g., linear interpolation or a higher order interpolation), the combination can be assigned an expected lifespan value that is based on the location of the point along a vertical line between the two curves. For example, in this example, the combination is assigned a value of 88 years in the reference table 250.

Referring back to FIG. 2A, determining the age adjustment factor 220 for users above a threshold age (45 years in this example) can include determining a standard estimated lifespan (215) which is independent of the glucose level. This can be done, for example, by accessing a standard life expectancy table 260 as shown in FIG. 2D. The example shown in FIG. 2D is a portion of the Actuarial Life Table published by the Social Security Administration in 2009, which provides a life expectancy of males and females between the ages of 0 and 119. The life expectancy provides the number of expected additional years of life for an individual of a particular age, and is computed based on, for example, a population size and corresponding death probability for the particular age. The life expectancy for a given age is added to the given age to obtain the expected lifespan based on the standard life expectancy table 260. For example, according to the data in the standard life expectancy table 260, the life expectancy of a 6 year old is 70.53 years. Therefore the expected lifespan, as obtained from the standard life expectancy table, is 76.53 years.

Referring again to FIG. 2A, the operations of the process 200 further includes determining the age adjustment factor 220 as a difference between the estimated lifespans obtained, for example, from the reference table 250 and the standard life expectancy table 260, respectively. If the estimated lifespan provided by the reference table 250 is higher than the standard life expectancy table 260, the age adjustment factor 220 is positive, and may indicate a gain in expected lifespan as compared to what is expected from the standard life expectancy table 260. On the other hand, if the estimated lifespan provided by the reference table 250 is lower than the standard life expectancy table 260, the age adjustment factor 220 is negative, and may indicate a loss in expected lifespan as compared to what is expected from the standard life expectancy table 260. In some implementations, the differences can be pre-computed and stored as the reference table 265, as shown in FIG. 2E.

In some implementations, the age adjustment factor 220 for a biomarker can also be computed based on one or more equations. In the example process 200, the age adjustment factor 220 for individuals less than 45 years old can be computed using a set of equations. The equations for computing the age adjustment factor can be determined, for example, from available data on mean glucose level as a function of age. FIG. 2F shows a plot 270 that includes exemplary curves representing mean glucose levels as a function of age separately for males and females. The plot 270 is based on data from the publication: Yashin, Anatoli I., Ukraintseva, Svetlana V., et al. “Maintaining physiological state for exceptional survival: What is the normal level of blood glucose and does it change with age?” Mechanisms of Ageing and Development. 130 (2009): 611-618. Equations for estimating age as a function of mean glucose level can be determined from such data. This can be done, for example, by re-plotting the data, switching the axes to interchange the dependent and independent variables, and fitting a line on the re-plotted data to identify a relationship between blood glucose level and age. The example plot 275 of FIG. 2G shows a re-plotted version of the data for males from the plot 270. A curve fitted on the re-plotted data of FIG. 2G can be represented by the equation:

y=−0.012x²+3.9097x−195.5

where y is age in years and x is blood glucose level in mg/dL. The equation for females in the present example is given by the equation:

y=−0.027x²+6.7019x−321.63.

In some implementations, the process 200 can include operations for determining an estimated age using the one or more equations (225). Once an estimated age for an individual is determined as a function of glucose level, the chronological age of the individual can be subtracted from the estimated age to determine an estimated number of years lost or gained due to the glucose level (230). In some implementations, if the number of years lost/gained is within a predetermined threshold or cap, the age adjustment factor 220 is represented using the number. In some implementations, if the age adjustment factor exceeds the threshold or cap, the threshold itself can be used as a capped age adjustment factor 221.

The threshold or cap value can be determined in various ways. In some implementations, diabetes can be used as a proxy for the maximum effect high glucose can have on lifespan. For example, available data on years lost due to diabetes can be used for determining the capped age adjustment factor 221. An example of such data is shown in the plot 280 of FIG. 2H. The exemplary plot 280 is based on data available in the publication: Morgan, Christopher L., Currie, Craig J., and John R. Peters. “Relationship Between Diabetes and Mortality” Diabetes Care. 23 (2000): 1103-1107. Such data can be re-plotted as illustrated in FIG. 21, with the number of years lost (y axis) being represented as a function of age (x axis). A curve can be fitted over the re-plotted points to obtain an equation that can be used to determine the age adjustment factor 221 as a function of age. In the example shown in FIG. 21, the equations for males and females are given by:

Males: y=154.3383e^{-0.0536x}

Females: y=185.8655e^{-0.0528x}

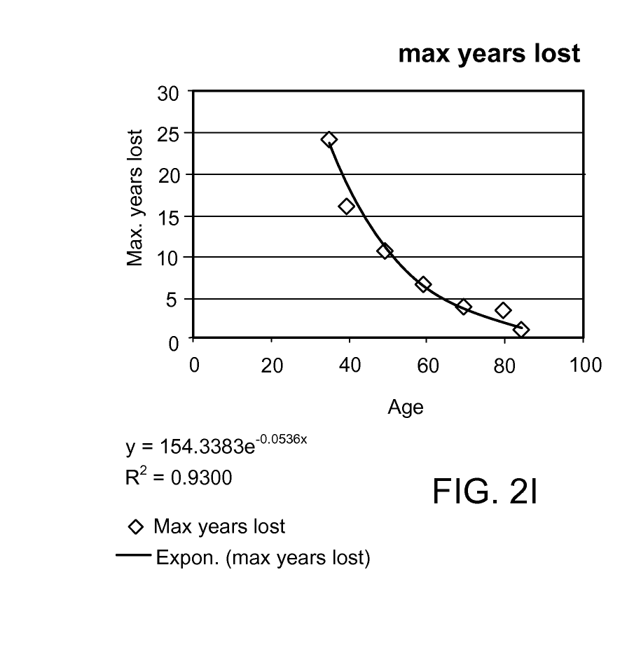

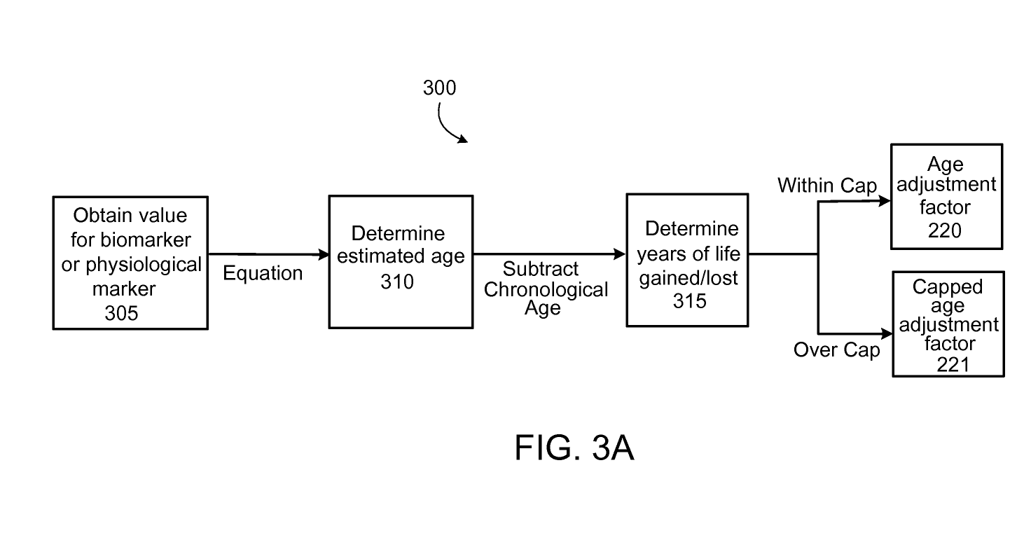

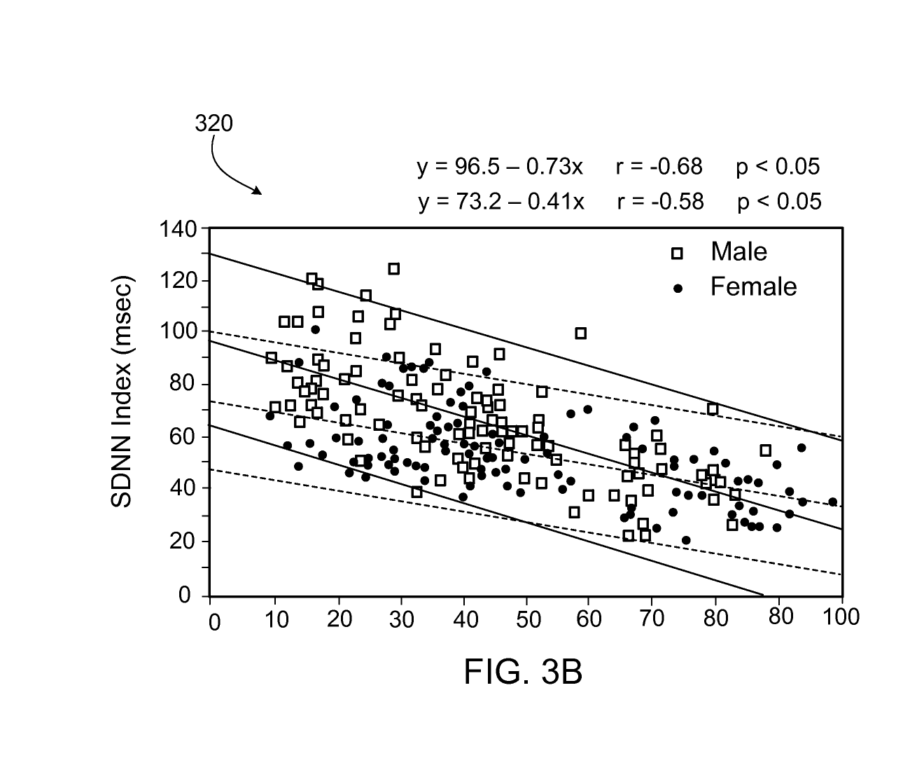

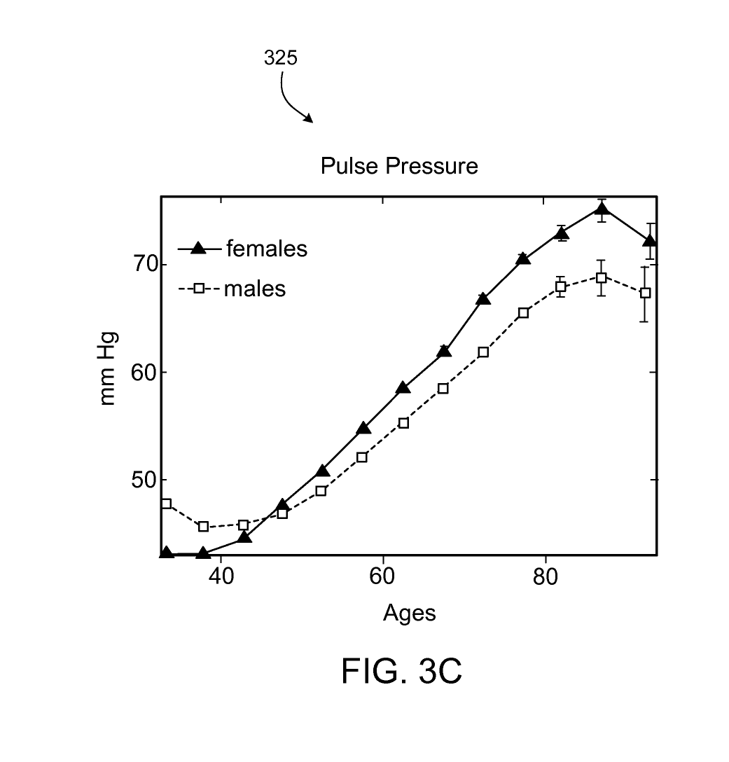

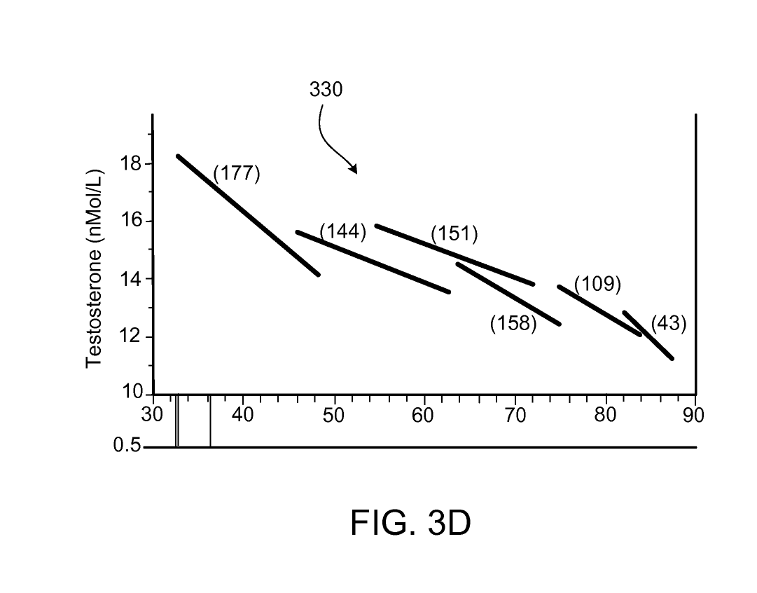

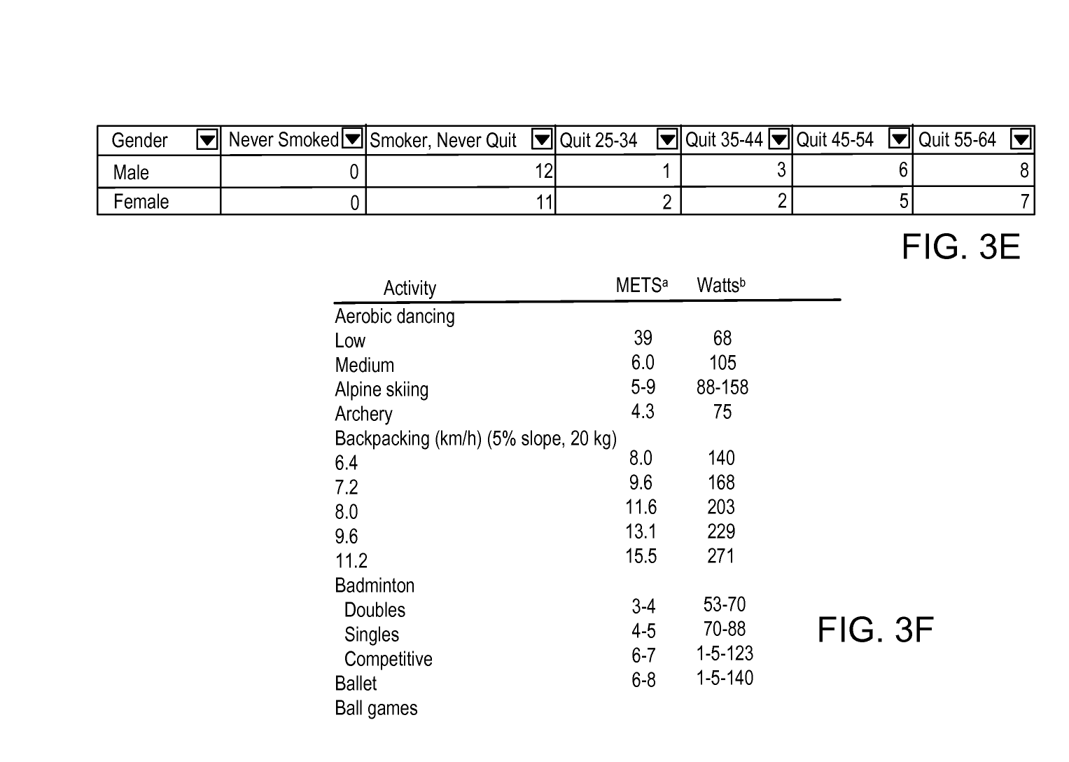

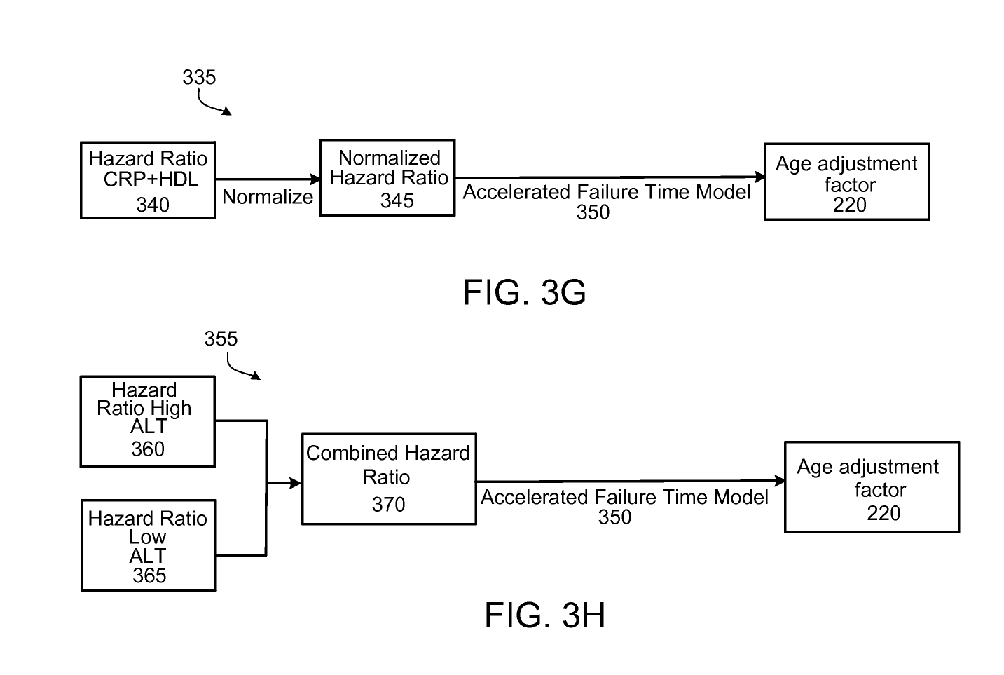

Age adjustment factors corresponding to other biomarkers and physiological markers can also be calculated using one or more equations. FIG. 3A shows a flowchart of an example process 300 that can be used in calculating an age adjustment factor 220 for a given biomarker or physiological marker. In some implementations, the example process 300 includes obtaining a value for the biomarker or physiological marker (305) for an individual. The value can be obtained, for example, using a device 105 or by other ways of obtaining a biomarker 110 or physiological marker 115 as discussed above with reference to FIG. 1. Other information about the individual (e.g., chronological age) is also obtained.

In some implementations, the process 300 can include determining an estimated age using one or more equations. The equations for computing the age adjustment factor 220 can be determined, for example, from collected data on the respective biomarker or physiological marker as a function of age. FIG. 3B shows a plot 320 that represents exemplary values for heart rate variability (HRV), represented by standard deviation of normal-to-normal (SDNN) intervals associated with heartbeats, as a function of age separately for males and females. The plot 320 is based on data from the publication: Umetani, K., Singer, Donald H., et al. “Twenty-Four Hour Time Domain Heart Rate Variability and Heart Rate: Relations to Age and Gender Over Nine Decades” Journal of the American College of Cardiology. 31(1998): 593-601. In another example, FIG. 3C shows a plot 325 that represents exemplary values for pulse pressure as a function of age separately for males and females. The plot 325 is based on data from the publication: Arbeev, Konstantin G., Ukraintseva, Svetlana V., et al. “Age trajectories of physiological indices in relation to healthy life course” Mech Ageing Dev. 132 (2011): 93-102. In yet another example, FIG. 3D shows a plot 330 that represents exemplary values for testosterone level in males as a function of age. The plot 330 is based on data from the publication: Harman, S. Mitchell, Metter, E. Jeffrey, et al. “Longitudinal Effects of Aging on Serum Total and Free Testosterone Levels in Healthy Men” The Journal of Clinical Endocrinology & Metabolism. 86 (2001): 724-731.

Equations for estimating age as a function of the various biomarkers and physiological markers can be determined from such data. This can be done, for example, by re-plotting the data, switching the axes to interchange the dependent and independent variables, and fitting a line on the re-plotted data to identify a relationship between the corresponding biomarker or physiological marker and age. The nature of the equations can be determined, for example, based on a quality of fit. Equations of various orders (e.g., linear, quadratic, cubic or higher order, logarithmic, or exponential) can be used. For example, a set of second order equations can be fitted, e.g., on re-plotted data from plot 320. These equations are:

Males: y=0.0303x²−7.2448x+400.45

Females: y=0.278x²−43.873x+1741.5

where y=age (yr), and x=HRV given as (20 ln rMSSD), and where rMSSD represents root mean squared successive differences between adjacent heartbeats. In another example, the set of equations corresponding to the plot 325 is:

Males: y=−0.0116x²+3.3288x−87.128

Females: y=−0.005x²+2.1281x−44.118

Where y=age (yr), x=pulse pressure (mmHg). The equation for testosterone based on the data of plot 330 is:

Males: y=0.0004x²−0.6767x+264.06

Where y=age (yr), x=testosterone (ng/dL).

In some implementations, the process 300 can include operations for determining an estimated age using the one or more equations (310). Once an estimated age for an individual is determined as a function of the level of biomarker or physiological marker, the chronological age of the individual can be subtracted from the estimated age to determine an estimated number of years lost or gained due to the corresponding biomarker or physiological marker (315). In some implementations, if the number of years lost/gained is within a predetermined threshold or cap, the age adjustment factor 220 is represented using the number. In some implementations, if the age adjustment factor exceeds the threshold or cap, the threshold itself can be used as a capped age adjustment factor 221.

The cap values for other biomarkers and physiological markers can also be set based on data collected through studies and research. For example, cap values for the physiological marker pulse pressure can be calculated by using hypertension as a proxy. Hypertension can be used, for example, to represent a maximum effect a high pulse pressure can have on lifespan to set the cap corresponding to pulse pressure at +/−2.7 years for men and +/−2.0 years for women. In some implementations, the cap values corresponding to HRV and testosterone can be selected as +/−2 years.

In some implementations, a corresponding age adjustment factor can be computed for one or more life style parameters 120 such as smoker status and level of athletic activities. The age adjustment factors for lifestyle parameters can be computed in various ways. For example, the age adjustment factor computed for smoking can depend on various parameters, e.g., if the person has ever smoked, if the person has quit, the time of quitting, number of cigarettes per day for active smokers etc. In some implementations, the age analysis engine 125 presents one or more of such queries to a user, for example, via a device 105, and computes an age adjustment factor, for example, based on collected data and research on effects of smoking on longevity. An example of such smoking-related data can be found in the following publication: Jha, Prabhat, Ramasundarahettige, Chinthanie, et al. “21st-Century Hazards of Smoking and Benefits of Cessation in the United States” The New England Journal of Medicine. 368 (2013): 341-350. Such smoking-related data can show the effects of smoking and quitting on expected lifespans, and the age analysis engine 125 can be configured to compute a corresponding age adjustment factor based on such data. In some implementations, the age analysis engine can be configured to estimate how many of the “lost” years can possibly be regained by ceasing smoking. For example, a man who begins smoking at age 25 and stops at age 40 may stand to regain 9 of the 12 years he is estimated to lose in comparison to a non-smoker.

In some implementations, the age adjustment factors for a given lifestyle parameter can be pre-computed and stored in a look-up table. Examples of such a look-up table is shown in FIG. 3E, which stores age adjustment factors as a function of gender and smoking status. In some implementations, data from such a look-up table can be stored in the database 130. The age analysis engine 125 can be configured to retrieve an appropriate age adjustment factor based on gender and smoking status data obtained by the age analysis engine 125.

For athletic activities, a corresponding age adjustment factor can be determined based on data on a type of athletic activity undertaken by a user. In some implementations, a corresponding score for each athletic activity can be obtained and a total score can be calculated for all the athletic activities. The individual scores corresponding to various activities can be stored, for example, in a reference table such as the one shown in FIG. 3F. The table shown in FIG. 3F is taken from the publication: Jette, M., Sidney, K., and G. Blumchen. “Metabolic Equivalents (METS) in Exercise Testing, Exercise Prescription, and Evaluation of Functional Capacity” Clinical Cardiology. 13 (1990): 555-565. In some implementations, each score can be multiplied by the amount of time spent performing the corresponding activity (e.g., in hr/wk), and the products added together to get the total score.

In some implementations, the total score can be adjusted based on one or more other parameters (e.g., BMI) to calculate the age adjustment factor corresponding to athletic activities. For example, adjustments to the total score can be a function of the BMI range of the user. The adjustments can be determined based on collected data and research on physical activity, such as found in the following publication: Moore, Steven C., Patel, Alpa C., et al. “Leisure Time Physical Activity of Moderate to Vigorous Intensity and Mortality: A Large Pooled Cohort Analysis”PLoS Medicine. 9 (2012). The data from this publication can be used to provide the following equations for computing BMI-dependent age adjustment factors:

BMI 18.5-24.9 y=−0.624x+4.7

BMI 25-29.9 y=−0.528x+3.9

BMI 30-34.9 y=−0.4587x+5

BMI 35+y=−0.3413x+7.2

where y=years of life lost, x=total score.

Age adjustment factors corresponding to biomarkers, physiological markers, and lifestyle parameters can be also be determined in other ways. For example, an age adjustment factor for BMI can be obtained from a look-up table that stores such factors, for example, as a function of age, gender and/or ethnicity. The look-up tables can be created and stored, for example, based on collected data and research, e.g., as provided in the publication: Fontaine, Kevin R., Redden, David T., et al. “Years of Life Lost Due to Obesity”American Medical Association. 289 (2003): 187-193. In some implementations, a hazard ratio may be computed for one or more combinations of biomarkers, physiological markers, and lifestyle parameters, and an age adjustment factor can be computed based on a statistical model such as the accelerated failure time model.

In some implementations, the age adjustment factors for vitamin D, ALT, or a combination of CRP and HDL can be computed using corresponding hazard ratios and accelerated failure time models. FIG. 3G shows a flowchart 335 for an example process used in calculating an age adjustment factor 220 using a hazard ratio 340 of a combination of CRP and HDL. In some implementations, a normalized hazard ratio 345 can be obtained from the hazard ratio 340, and the age adjustment factor 220 can be computed using the normalized hazard ratio. For these markers, an accelerated failure time model 350 can be used to derive age adjustment factors, for example, from the normalized hazard ratio 345, for known demographic data. In some implementations, a fitted curve, such as a polynomial fit can be used to map changes in hazard ratio vs. marker level to changes in years of life lost or gained vs. marker level. Computation of hazard ratios for biomarkers such as vitamin D is described, for example, in the publication: Durup, D., Jorgensen, H. L., et al. “A Reverse J-Shaped Association of All-Cause Mortality with Serum 25-Hydroxyvitamin D in General Practice: The CopD Study” The Journal of Endocrinology and Metabolism. 97(2012): 2644-2652. The effect of CRP on mortality is described, for example, in the publication: Kim, Kwang-II, Oh, Se W., et al. “CRP Level and HDL Cholesterol Concentration Jointly Predict Mortality in a Korean Population” The American Journal of Medicine. 125 (2012): 787-795.

FIG. 3H shows a flowchart 355 for an example process used in calculating an age adjustment factor 220 from hazard ratios 360 and 365 corresponding to high ALT and low ALT, respectively. In some implementations, the process includes computation of a combined hazard ratio 370 from the hazard ratios 360 and 365. An accelerated failure time model 350 can then be used to compute the age adjustment factor 220 from the combined hazard ratio 370. Computation of hazard ratios for ALT is described, for example, in the publication: Lee, Tae H., Kim, W. R., et al. “Serum Aminotransferase Activity and Mortality Risk in a United States Community” Hepatology. 47 (2008): 880-887, and in the publication: Ford, Ian, Mooijaart, Simon P., et al. “The inverse relationship between alanine aminotransferase in the normal range and adverse cardiovascular and non-cardiovascular outcomes” International Journal of Epidemiology. 40 (2011) 1530-1538.

The age analysis engine 125 can be configured to compute a combined age adjustment factor from the individual age adjustment factors corresponding to the various biomarkers, physiological marker, and lifestyle parameters. In some implementations, the combined age adjustment factor can be computed as a sum of the individual age adjustment factors. In some implementations, simply summing the individual age adjustment factors overestimates the total combined effect of markers. For example, if expected lifespan is lost due to the effect of a single marker, the potential loss from another marker may be decreased. In such cases, the individual age adjustment factors can be combined using a model that is monotonic between zero and a maximum possible number of years lost or gained.

Using such a model, a maximum life change (MLC) can be defined as (estimated unmodified age at death)−(current age) for years lost, and as (18-current age) for years gained (assuming a minimum age for individuals as 18). The quantity SUM can be defined as the sum of the individual age adjustment factors corresponding to the various biomarkers, physiological markers, and lifestyle parameters. From this a combined age adjustment factor can be determined as:

$$MLC = ( 1 - {e}^{\frac{- SUM}{( MLC * k )}} )$$

where k is an adjustable parameter that can be adjusted to alter the steepness of the monotonic function associated with the model.

The age analysis engine 125 is further configured to add the combined age adjustment factor to the chronological age of the individual to provide the physiological age 135. In some implementations, the age analysis engine can be configured to also provide personalized recommendations 140 (e.g., a computer-implemented and web-based personalized nutrition and exercise program) based on the determined physiological age. The selected combination biomarkers, physiological markers and lifestyle parameters may also provide indications on normal or impaired biological processes in response to food, lifestyle, or exercise interventions. In some implementations, a web-based application can provide a nutrition, supplement, lifestyle, and physical training regime personalized to improving a user's estimated physiological age. In some implementations, the age analysis engine 125 may also be configured to provide a ranking of the various markers' contributions to accelerating or decelerating the physiological age. The age analysis engine can also be configured to prioritize recommendations based on information that is likely to improve the physiological age.

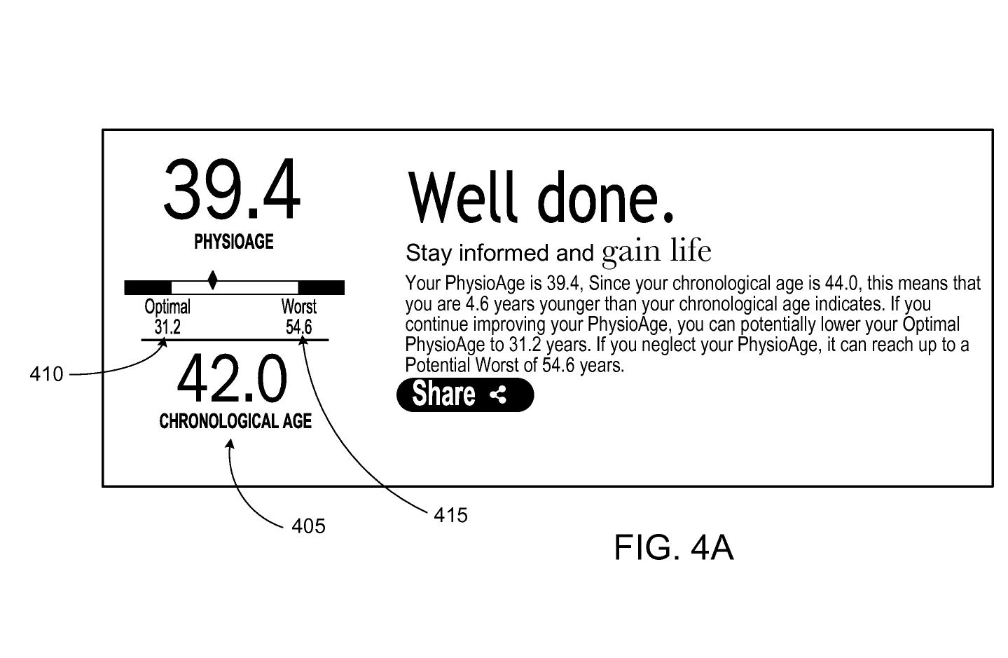

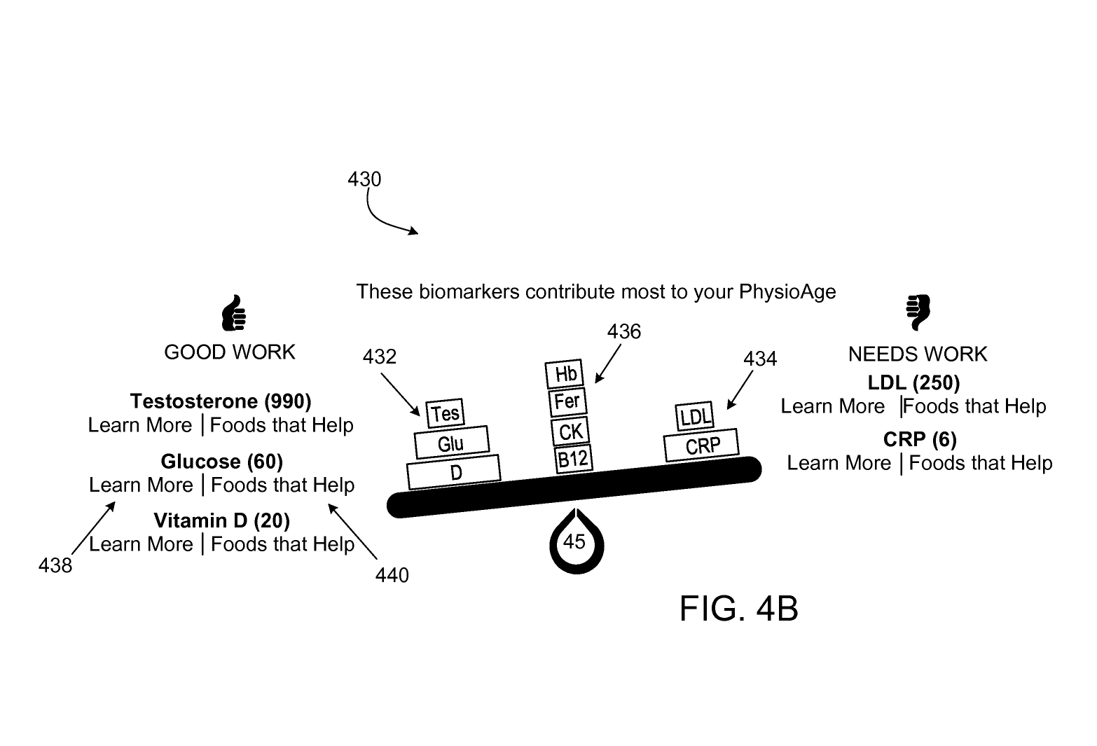

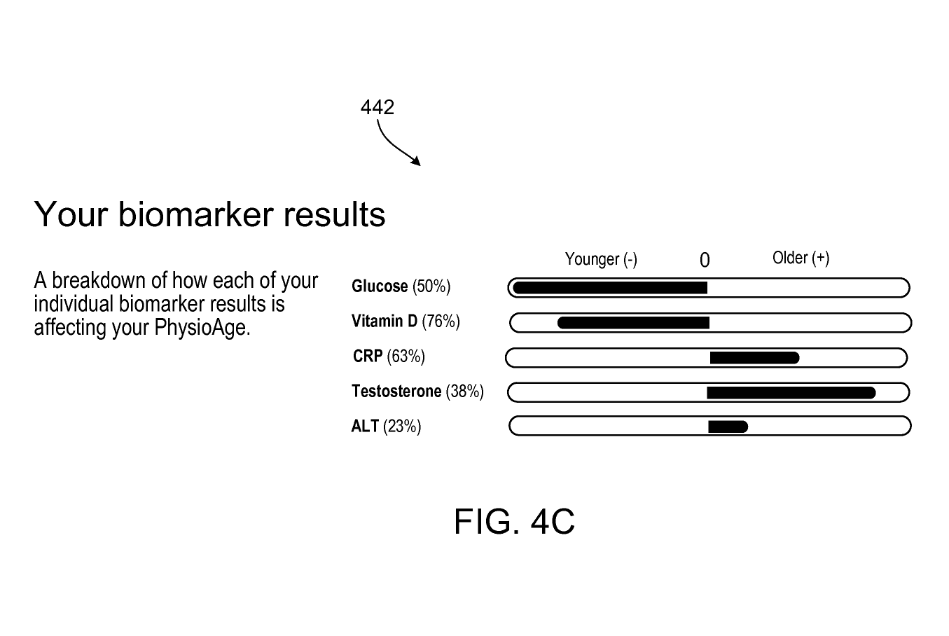

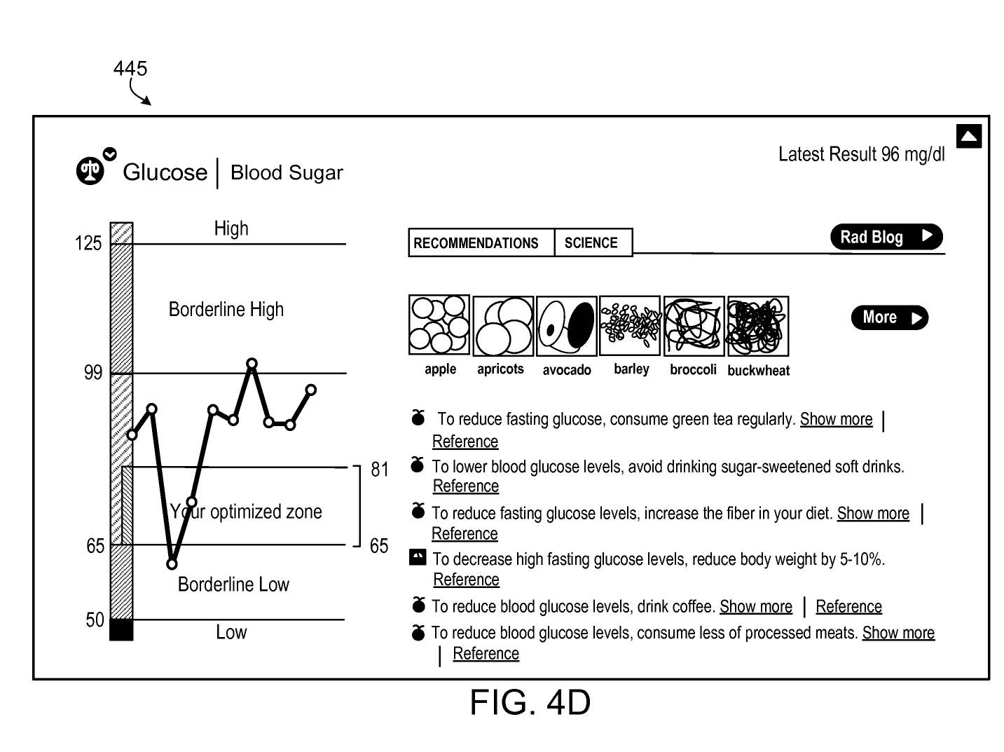

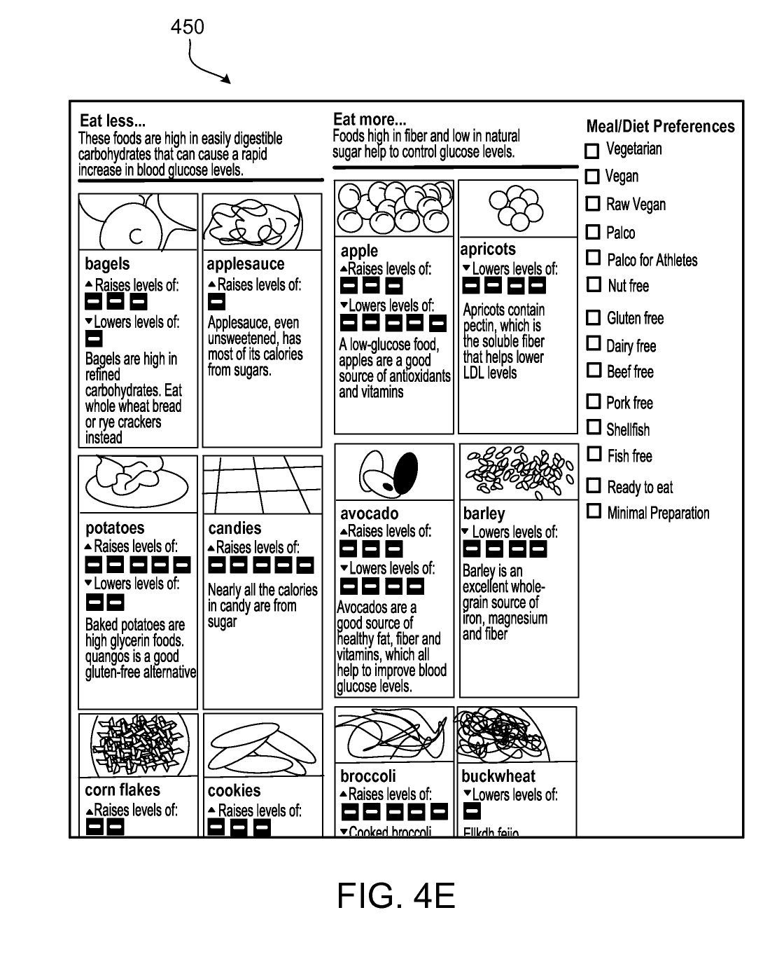

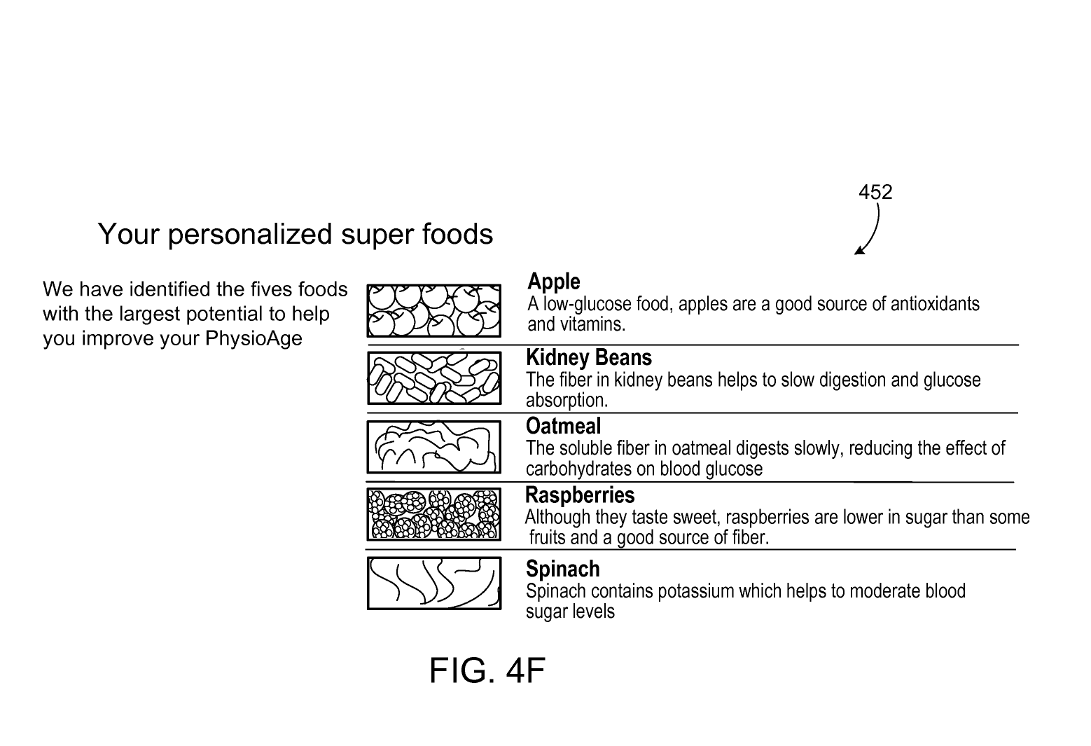

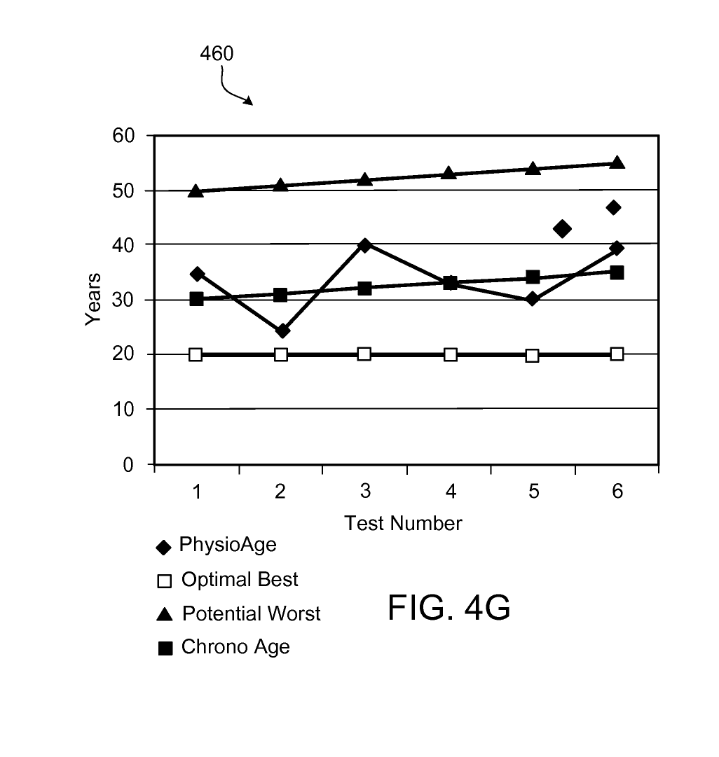

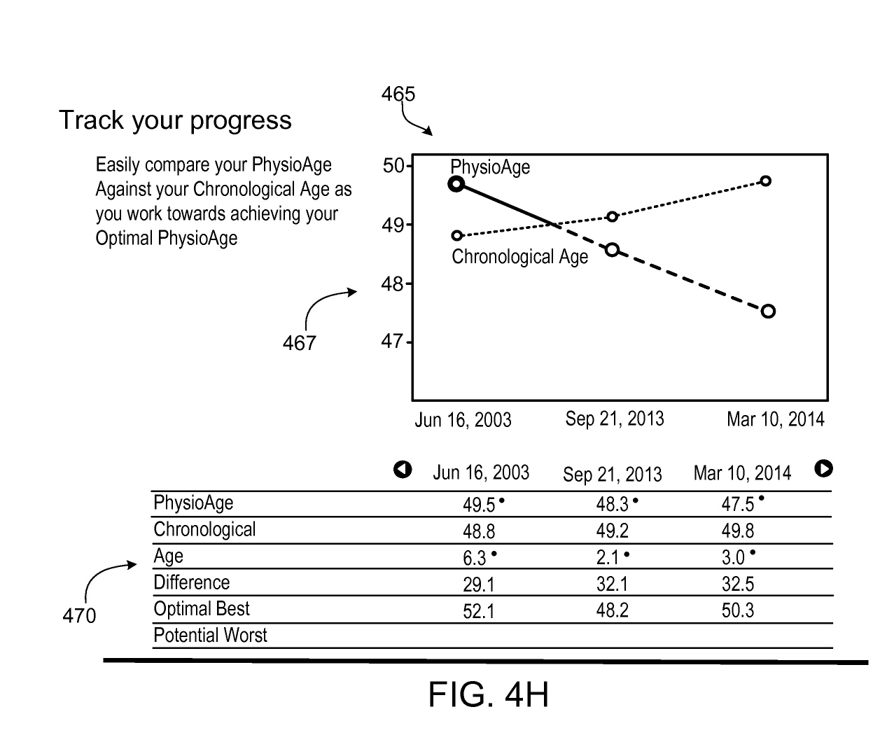

In some implementations, the age analysis engine can also be configured to determine an upper and lower limit for a physiological age possible for the individual, and a ranking of the individual marker contributions. Such upper and lower limits can be a function of, for example, age and gender. To calculate an upper limit for the physiological age, worst case marker values (e.g., for the corresponding age and gender) for each marker can be considered to calculate the physiological age. To calculate the lower limit for the physiological age, the most favorable marker values (e.g., for the corresponding age and gender) may be used for the markers. The physiological age 135 and the corresponding upper and lower limits can be displayed to a user via a graphical user interface such as the interface 400 shown in FIG. 4A. In the example of FIG. 4A, the interface 400 is configured to display the determined physiological age 402 together with the user's chronological age 405, the lower limit 410, and the upper limit 415.

The ranking of each marker's contribution can be derived, for example, from a breakdown of how many years each marker adds to or subtracts from the physiological age. Such a ranking system allows the user to prioritize on improving marker levels to focus on markers that are more likely to improve their physiological age. In some implementations, the ranking of the various biomarkers and physiological markers can be displayed, for example, using a graphical user interface such as the interface 430 shown in FIG. 4B. In the example of FIG. 4B, the interface 430 includes a graphical representation of the markers 432 (and their rankings) that are contributing in decreasing the user's physiological age, a graphical representation of the markers 434 (and their rankings) that are contributing in increasing the users physiological age, and a graphical representation of the markers 436 that are at expected levels. The interface 430 can also include controls (e.g., hyperlinks) for accessing more information on the markers and tips (e.g., list of foods or actions) for improving/maintaining the levels of corresponding markers. For example, the control 438, upon activation (e.g., by a click or selection), may cause a display of another interface (e.g., the interface 445, as shown in FIG. 4D) that displays more information on the corresponding marker (glucose, in this example). Other types and forms of graphical user interfaces can also be used for displaying the results corresponding to the various biomarkers/physiological markers. For example, the ranking of the various biomarkers and physiological markers can also be displayed using an alternative graphical user interface such as the interface 442 shown in FIG. 4C.

As noted above, the methods can include providing recommendations to the individual for improving the levels of the biochemical or physiological markers, e.g., for altering or improving (e.g., reducing) the subject's physiological age. In some implementations, the age analysis engine 125 can be configured to provide personalized recommendations 140 to improve levels of one or more biochemical or physiological markers. These recommendations can include, for example, recommendations on nutrition, supplementation, and lifestyle changes. In some implementations, activating the control 440 within the interface 430 can cause a display of another interface (e.g., the interface 450 of FIG. 4E or the interface 452 of FIG. 4F) that includes the recommendations 140. The interface 450 can include, for example, recommendations related to foods associated with a particular biomarker or physiological markers. The recommendations can indicate foods to avoid as well as foods to consume. The recommendations can be made specific to the user's results (i.e., based on the user's biomarker levels and/or physiological markers), and can be configured to help the user improve levels of components that negatively contribute to the physiological age, while maintaining levels of components that positively contributing to the physiological age.

In some implementations, the recommendations can include a set of food items that are expected to improve the physiological age of the user. An example of an interface 452 that provides such recommendations is illustrated in FIG. 4F. The food items to be included in the set can be determined by the age analysis engine 125 based on the results corresponding to the various biomarkers/physiological markers of the user. In some implementations, a predetermined number of food items (e.g., between three and ten items) can be selected from a database of various food items. The database can be configured to store information on how the various food items affect the levels of biomarkers/physiological markers for different groups of individuals (e.g., men, women, children, athletes, active individuals, etc.). The predetermined number of food items can be selected by the age analysis engine such that, for example, the selected food items are expected to improve at least one or more, e.g., at least two, at least three, out-of-range biomarkers/physiological markers, preferably without adversely affecting the levels of other biomarkers/physiological markers. For example, if a user has high glucose and high CRP, the age analysis engine 125 can be configured to select food items that lower glucose levels without increasing or otherwise adversely affecting the levels of CRP for the user. In some implementations, the age analysis engine 125 can be configured to select food items that are expected to lower both glucose levels and CRP levels. On the other hand, if a particular food item is known to lower glucose levels and increase CRP levels, the age analysis engine can be configured to prevent selection of such a food item for an individual with high glucose and CRP levels. However, the age analysis engine may pick that particular food item for another user who has elevated glucose levels and diminished CRP levels.

The food items can be selected, for example, based on the presence of one or more nutrients in the food items that improve out-of-range biomarkers or physiological markers. The nutrients can be correlated to a biomarker/physiological marker of interest based on, for example, information stored in a database. The information in the database can be obtained, for example, by mining available scientific literature documents on nutrients and biomarkers/physiological markers. In some implementations, the database can be created and/or updated using various data-mining processes on electronically stored versions of such scientific literature documents. The scientific literature documents can be selected based on, for example, a choice of population, a clinical trial design, or and statistical significance of obtained results. The data obtained from the scientific literature documents can include studies that test effect of nutrients in the form of food or supplements.

In some implementations, information on nutrients that are determined to be beneficial for one or more biomarkers and/or physiological markers are stored in a food database. The information stored in the food database can be used on selecting the one or more food items expected to improve the physiological age of the user. In some implementations, standard databases related to foods can also be used for this purpose. For example, a database such as the USDA Standard Reference Database can be used for selecting the food items. The USDA database includes information on macronutrient and micronutrients associated with a large variety of food items. In some implementations, the reference database can be pre-processed to remove redundant food items or consolidate several food items as one entry. For example, one or more rules can be created and applied to various food groups (e.g. nut and seed product) and food categories (e.g. peanuts and tree nuts) to remove redundant entries, without biasing the database toward any particular food or food group.

In some implementations, foods that cannot be consumed in sufficiently large quantities to have an effect on the physiological age of the subject (e.g., herbs and spices) may be removed or excluded from the food database used in selecting the recommended food items. The amount of nutrients can then be calculated by normalizing with respect to serving sizes. This can allow for comparing nutrients across food items that are typically consumed in different serving sizes. For example, a typical serving size for almonds can be 30 gram, whereas a typical serving size for salmon can be 150 gram. Normalizing with respect to serving sizes therefore allows for accurate nutrient comparisons across various food items. For example, an appropriate serving size can be applied to calculate the food items' nutrient contents (for example, by multiplying a given nutrient content by the adjusted serving size and dividing by 100).

In some implementations, various food items affecting a particular biomarker or physiological marker can be ranked based on, for example, amounts of a given nutrient in the food item. The ranked food items can then be stored in the food database. For example, the highest rank (e.g., a rank of 1) can be given to the food item including the highest amount of the given nutrient. In some implementations, the highest rank can be given to the food with an ideal amount of the given nutrient. The ideal amounts can be determined, for example, based on the information from the scientific literature and/or the % DV of the given nutrient, determined, for example, using the FDA's Guidance for Industry: A Food Labeling Guide. In some cases, food items can be ranked only if they contain sufficient amounts of the given nutrient for the given biomarker. On the other hand, a food item that has little to no amount of the given nutrient can be assigned a low rank. The assigned ranks can then be added across potential combinations of biomarkers (for example, a combination of two, three or more out-of-range markers). For example, if glucose and testosterone are both out of range, a food item that improves both markers can be suggested. For that purpose a food item that has a high rank for both markers can be selected.

In some implementations, the suggested food items can be selected by the age analysis engine, for example, based on food types. For example, a combination of a predetermined number of natural and processed food items can be selected for each combination of out-of-range markers. For example, a combination of five natural food items and one processed food item can be suggested for a combination of out-of-range markers. The natural food items can be selected from a variety of food groups. For example, instead of selecting five different types of beans, the age analysis engine 125 can be configured to choose a combination of beans, vegetables, and meats.

In some implementations, the food groups can be identified, for example, based on an identifier assigned to the food items for which information is stored in the food database. For example, the food items can be assigned numeric identifiers and the first predetermined number of digits may identify corresponding food groups for the food items. In some implementations, the food groups may be further divided into sub-categories. For example, potatoes and spinach may both be classified into the food group vegetables, but may be divided into sub-categories corresponding to tubers and leaves, respectively. For situations where food items from a variety of food groups are not available for a biomarker combination, a predetermined number (e.g., five) of food items that are ranked the highest for that combination may be selected.

In some implementations, the food database can include at least a predetermined number of categories. In one example, the food database includes four categories (also referred to as tiers), in total. The predetermined number of categories can be determined, for example, as the minimum number of categories that yields diverse food options for various combinations of out of range biomarkers. In some implementations, a set of rules can be established to ensure that a variety of foods are selected by a processing device. The set of rules can be specific to, for example, the different tiers or categories. For example, the rules for one particular tier may allow one particular food type (e.g., grain) in two food items, whereas for another tier, no two food item names could be the same. Under these example rules, barley and oats could be selected together, but wheat bagel and onion bagel would not be selected together in a single set of food recommendations.

In some implementations, the age analysis engine 125 can be configured to select food items based on other criteria. For example, food items can be selected based on availability information associated with the food items. Such availability information can also be stored in the food database. For example, a high ranked food item may be omitted from a selection based on determining that the food item is not easily available at the location of the user. For example, if a food item is known to be not easily available in a particular country, the food item may be omitted from being selected for a user located in that particular country (even if the food item is determined to be highly beneficial for a combination of out-of-range markers for the user). In such cases, if a difficult to purchase food item (e.g., mamey) is ranked higher than an easy to purchase comparable food item (e.g., orange), the latter food item may be selected for recommendation over the former. Availability information for food items can be determined, for example, based on availability at non-specialty grocery stores.

In some implementations, the age analysis engine 125 can be configured to select food items based also on one or more user-defined preferences. For example, if a user indicates a dislike for a particular food item, the age analysis engine 125 can be configured to omit the particular food item from a selection even if the particular food item is determined to be beneficial for the user. Dietary preferences (vegetarian, gluten-free etc.), as well as religion or culture based preferences (if available) may also be taken in to account by the age analysis engine 125 in selecting the food items for recommendation.

In some implementations, the age analysis engine 125 can also provide an interface to track progress of a user. An example of such an interface 460 is shown in FIG. 4G. The interface 460 can be configured to display variations in physiological age in comparison to the increasing chronological age, as a function of tests or measurements of the markers over time. In some implementations, the interface 460 is also configured to display the variations in the upper and lower limits of the physiological age as functions of the tests or measurements of the markers over time. Another example of an interface 465 that can be used for tracking progress is shown in FIG. 4H. Such an interface can be used to provide, for example, one or more of a graphical representation 467 that shows the changes in estimated physiological age over time, and a table 470 that provides the corresponding details.

The exemplary user interfaces illustrated in FIGS. 4A-4H can be displayed on a device 105 of the user. In some implementations, a user may be able to log in to a web-based account via a website or an application installed on a phone or tablet to view results and personalized recommendations. Such recommendations can help the user review levels of various biomarkers and physiological markers and take appropriate steps to improve such levels. This is illustrated using the following examples.

In one example, an apparently healthy 25-year-old male of typical height and weight is tested for a set of biomarkers and physiological markers, and it is determined that he is not as healthy as he appears. For example, he has high glucose, high ALT from alcohol consumption, and although he has a normal BMI, his activity level indicates inadequate exercise. Based on this data, the age analysis engine 125 may determine that his physiological age is 35. The personalized recommendations provided by the age analysis engine can include, for example, reducing alcohol consumption to lower ALT, increasing daily amount of exercise, and consuming foods that promote a reduction of blood glucose level. After following these recommendations for 6 months, the person may be retested for the biomarkers and physiological markers to determine that his physiological age has been reduced to 30.

In some implementations, the physiological age of a person may be determined based on a limited set of data on biomarkers, physiological markers, and lifestyle parameters. In such cases, the age analysis engine 125 can be configured to determine the individual contribution of each available marker while ignoring the markers for which data is unavailable. For the same 25-year-old male from the previous example, if the age analysis engine 125 receives data on the high glucose and high ALT level, but not on the low activity level, the physiological age may be determined to be 33 instead of 35.

In some implementations, the age analysis engine 125 can be configured to estimate a user's physiological age based on the data on the user's glucose, vitamin D, and CRP levels. Other biomarkers, physiological markers and lifestyle parameters can be used to increase the accuracy of the estimate. For example, the accuracy can be increased if other biomarkers such as ALT and testosterone, and/or other physiological markers such as one or more of BMI, pulse pressure, HRV, and/or lifestyle parameters such as activity level and smoking are also taken into account in calculating the physiological age.

In some implementations, integration of feedback from connected sensors (e.g., wearable devices configured to measure one or more of biomarkers, physiological markers, and lifestyle parameters) may allow frequent updates and feedback related to the estimated physiological age. For example, fitness monitors can be used to obtain information about physical activity, digital scales can be used for measuring BMI, and HRV monitors and blood pressure cuffs can be used to obtain HRV and pulse pressure information. As the physiological age is updated based on feedback from one or more sensors or devices, the recommendations for the user can also be updated.

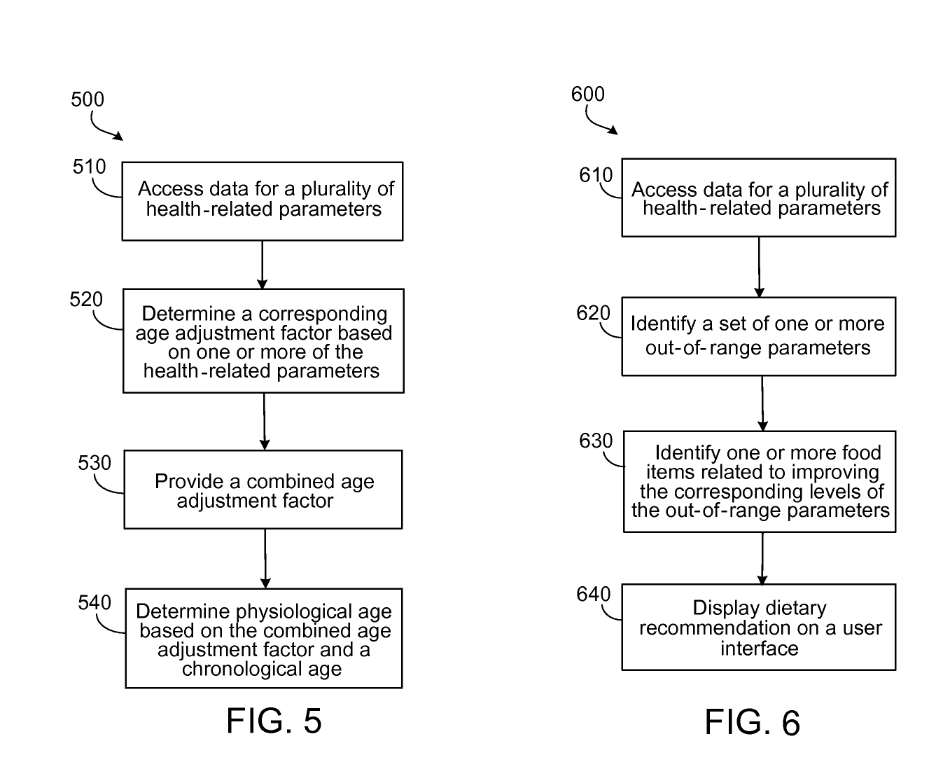

FIG. 5 shows a flowchart 500 for an example process for determining a physiological age of an individual. The process shown in FIG. 5 can be executed, for example, by one or more processors of the age analysis engine 125 described with reference to FIG. 1. The process includes accessing data for a plurality of health-related parameters of an individual (510). The data for the plurality of health-related parameters can include, for example, data on levels of one or more biomarkers (e.g., glucose, testosterone, vitamin D, C-CRP, or ALT), data on levels of one or more physiological markers (e.g., HRV, pulse pressure, or BMI), and data on one or more lifestyle-related parameters such as smoking habits or activity levels.

The process can also include determining a corresponding age adjustment factor based on one or more of the health-related parameters (520). This can be done, for example, using any of the above-described processes for calculating the age adjustment factors 220. For example, an age adjustment factor for glucose can be calculated using the process described with reference to FIG. 2. The age adjustment factors can also be calculated using the processes described with reference to FIG. 3A, 3G or 3H. For example, determining an individual age-adjustment factor for a particular health related parameter can include determining an estimated age of the individual based on the data for the particular health-related parameter, and determining the individual age adjustment factor as a difference between the chronological age and the estimated age. The estimated age can be determined, for example, using an equation that relates the particular health parameter to the estimated age.

In some implementations, determining an individual age-adjustment factor for a particular health related parameter can also include estimating a projected lifespan for the individual based on the chronological age of the individual as well as the data for the particular health-related parameter. In some implementations, a standard expected lifespan for the individual can be determined based on accessing life-expectancy data for a population of which the individual is a part. The individual age adjustment factor can then be determined as a difference between the projected lifespan and the standard expected lifespan. The projected lifespan can be estimated, for example, by accessing a look-up table (e.g., an actuarial life table) that stores projected lifespan values as a function of tuples that include the chronological age of the individual and the data for the particular health-related parameter. An example of such a look-up table is shown in FIG. 2B.

The process can also include providing a combined age adjustment factor from the individual age-adjustment factors computed for the various health related parameters (530). The combined age adjustment factor can be computed, for example, as described above with reference to the age analysis engine 125. The process also includes determining a physiological age for an individual based on both the combined age adjustment factor and the chronological age of the individual (540). This can be done, for example, by adjusting the chronological age by adding or subtracting a number of years based on the combined age adjustment factor.

FIG. 6 shows a flowchart 600 for an example process for providing dietary recommendations to an individual. The process shown in FIG. 6 can be executed, for example, by one or more processors of the age analysis engine 125 described with reference to FIG. 1. In some implementations, the dietary recommendations can be provided by a recommendation engine that may be implemented as a part of the age analysis engine, or as a module external to and/or independent of the age analysis engine 125.

The process includes accessing data for a plurality of health-related parameters of an individual (610). The data for the plurality of health-related parameters can include, for example, data on levels of one or more biomarkers (e.g., glucose, testosterone, vitamin D, C-CRP, or ALT), data on levels of one or more physiological markers (e.g., HRV, pulse pressure, or BMI), and data on one or more lifestyle-related parameters such as smoking habits or activity levels.

The process also includes identifying a set of one or more out-of-range parameters (620). The out-of-range parameters can be identified, for example, based on accessing a database that stores data on normal or acceptable ranges for the various health related parameters. Normal or acceptable ranges can be stored in the database, for example, for various combinations of parameters such as age, gender, ethnicity, activity level etc.

The process further includes identifying one or more food items related to improving the corresponding levels of the out-of-range parameters (630). The one or more food items can be identified, for example, using the various techniques described above with reference to FIGS. 4D-4F. The identified food items can include one or more food items to consume and/or one or more food items to avoid. The one or more food items can be identified based on information on various candidate food items stored in a food database. In some implementations, the food database includes information on nutrients such as macronutrients and micronutrients associated with the food items. Identifying the one or more food items can include identifying one or more nutrients known to affect the set of one or more out-of-range parameters, and comparing a set of candidate food items based on corresponding normalized amounts of the one or more nutrients in the candidate food items. The normalization can be done, for example, with respect to predetermined serving sizes corresponding to the candidate food items. The one or more food items can then be selected from the candidate food items based on the comparison.

The process can also include displaying the dietary recommendation on a user interface (640). Examples of such a user interface are described above with reference to FIGS. 4E and 4F. The user interface can be presented on a display device associated with a computing device such as a laptop or desktop computer, or a mobile device.

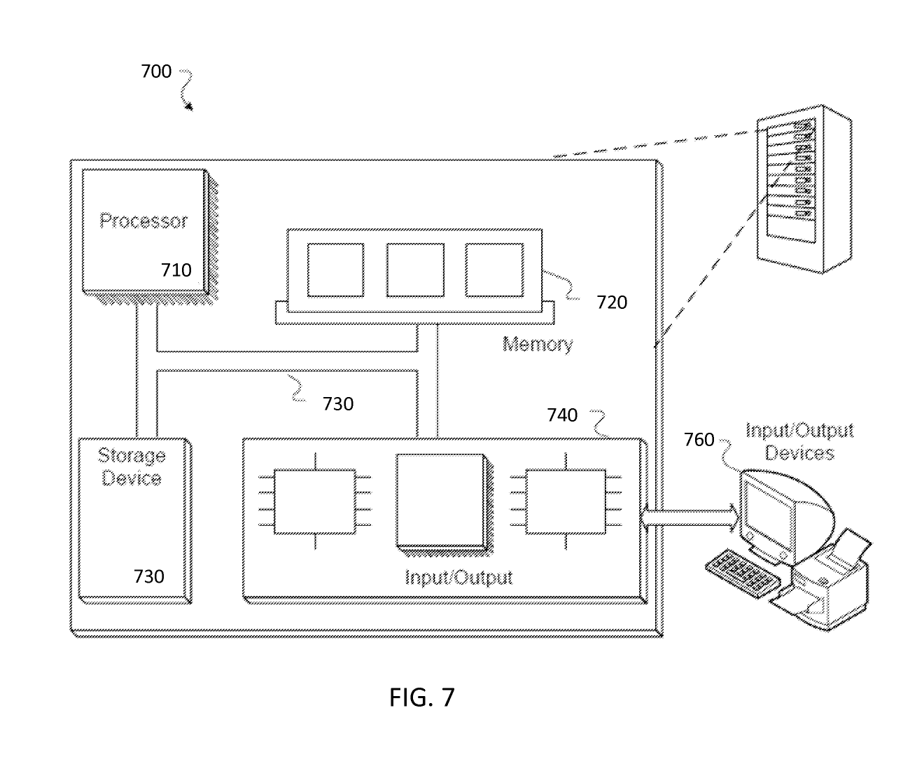

FIG. 7 is block diagram of an example computer system 700 that may be used in performing the processes described herein. For example, the age analysis engine 125, and the devices 105 described above with reference to FIG. 1, can include at least portions of the computing device 700 described below. Computing device 700 is intended to represent various forms of digital computers, such as laptops, desktops, workstations, servers, blade servers, mainframes, and other appropriate computers. Computing device 700 is further intended to represent various typically non-mobile devices, such as televisions or other electronic devices with one or more processers embedded therein or attached thereto. Computing device 700 also represents mobile devices, such as personal digital assistants, touchscreen tablet devices, e-readers, cellular telephones, and smartphones.

The system 700 includes a processor 710, a memory 720, a storage device 730, and an input/output module 740. Each of the components 710, 720, 730, and 740 can be interconnected, for example, using a system bus 750. The processor 710 is capable of processing instructions for execution within the system 700. In one implementation, the processor 710 is a single-threaded processor. In another implementation, the processor 710 is a multi-threaded processor. The processor 710 is capable of processing instructions stored in the memory 720 or on the storage device 730.

The memory 720 stores information within the system 700. In one implementation, the memory 720 is a computer-readable medium. In one implementation, the memory 720 is a volatile memory unit. In another implementation, the memory 720 is a non-volatile memory unit.

The storage device 730 is capable of providing mass storage for the system 700. In one implementation, the storage device 730 is a computer-readable medium. In various different implementations, the storage device 730 can include, for example, a hard disk device, an optical disk device, or some other large capacity storage device.

The input/output module 740 provides input/output operations for the system 700. In one implementation, the input/output module 740 can include one or more of a network interface devices, e.g., an Ethernet card, a serial communication device, e.g., an RS-232 port, and/or a wireless interface device, e.g., and 802.11 card. In another implementation, the input/output device can include driver devices configured to receive input data and send output data to other input/output devices, e.g., keyboard, printer and display devices 760.

The web server, advertisement server, and impression allocation module can be realized by instructions that upon execution cause one or more processing devices to carry out the processes and functions described above. Such instructions can comprise, for example, interpreted instructions, such as script instructions, e.g., JavaScript or ECMAScript instructions, or executable code, or other instructions stored in a computer readable medium. The web server and advertisement server can be distributively implemented over a network, such as a server farm, or can be implemented in a single computer device.

Example computer system 700 can include a server. Various servers, which may act in concert to perform the processes described herein, may be at different geographic locations, as shown in the figure. The processes described herein may be implemented on such a server or on multiple such servers. As shown, the servers may be provided at a single location or located at various places throughout the globe. The servers may coordinate their operation in order to provide the capabilities to implement the processes.

Although an example processing system has been described in FIG. 7, implementations of the subject matter and the functional operations described in this specification can be implemented in other types of digital electronic circuitry, or in computer software, firmware, or hardware, including the structures disclosed in this specification and their structural equivalents, or in combinations of one or more of them. Implementations of the subject matter described in this specification can be implemented as one or more computer program products, e.g., one or more modules of computer program instructions encoded on a tangible program carrier, for example a non-transitory computer-readable medium, for execution by, or to control the operation of, a processing system. The non-transitory computer readable medium can be a machine readable storage device, a machine readable storage substrate, a memory device, or a combination of one or more of them.

In this regard, various implementations of the systems and techniques described herein can be realized in digital electronic circuitry, integrated circuitry, specially designed ASICs (application specific integrated circuits), computer hardware, firmware, software, and/or combinations thereof. These various implementations can include implementation in one or more computer programs that are executable and/or interpretable on a programmable system including at least one programmable processor, which can be special or general purpose, coupled to receive data and instructions from, and to transmit data and instructions to, a storage system, at least one input device, and at least one output device.

These computer programs (also known as programs, software, software applications or code) include machine instructions for a programmable processor, and can be implemented in a high-level procedural and/or object-oriented programming language, and/or in assembly/machine language. As used herein, the terms “machine-readable medium” “computer-readable medium” refers to a computer program product, apparatus and/or device (e.g., magnetic discs, optical disks, memory, Programmable Logic Devices (PLDs)) used to provide machine instructions and/or data to a programmable processor, including a machine-readable medium that receives machine instructions as a machine-readable signal. The term “machine-readable signal” refers to signal used to provide machine instructions and/or data to a programmable processor.

To provide for interaction with a user, the systems and techniques described here can be implemented on a computer having a display device (e.g., a CRT (cathode ray tube) or LCD (liquid crystal display) monitor) for displaying information to the user and a keyboard and a pointing device (e.g., a mouse or a trackball) by which the user can provide input to the computer. Other kinds of devices can be used to provide for interaction with a user as well; for example, feedback provided to the user can be a form of sensory feedback (e.g., visual feedback, auditory feedback, or tactile feedback); and input from the user can be received in a form, including acoustic, speech, or tactile input.

The systems and techniques described here can be implemented in a computing system that includes a back end component (e.g., as a data server), or that includes a middleware component (e.g., an application server), or that includes a front end component (e.g., a client computer having a graphical user interface or a Web browser through which a user can interact with an implementation of the systems and techniques described here), or a combination of such back end, middleware, or front end components. The components of the system can be interconnected by a form or medium of digital data communication (e.g., a communication network). Examples of communication networks include a local area network (“LAN”), a wide area network (“WAN”), and the Internet.

The computing system can include clients and servers. A client and server are generally remote from each other and typically interact through a communication network. The relationship of client and server arises by virtue of computer programs running on the respective computers and having a client-server relationship to each other.

Content, such as ads and GUIs, generated according to the processes described herein may be displayed on a computer peripheral (e.g., a monitor) associated with a computer. The display physically transforms the computer peripheral. For example, if the computer peripheral is an LCD display, the orientations of liquid crystals are changed by the application of biasing voltages in a physical transformation that is visually apparent to the user. As another example, if the computer peripheral is a cathode ray tube (CRT), the state of a fluorescent screen is changed by the impact of electrons in a physical transformation that is also visually apparent. Moreover, the display of content on a computer peripheral is tied to a particular machine, namely, the computer peripheral.

For situations in which the systems and methods discussed here collect personal information about users, or may make use of personal information, the users may be provided with an opportunity to control whether programs or features that may collect personal information (e.g., information about a user's calendar, social network, social actions or activities, a user's preferences, or a user's current location), or to control whether and/or how to receive content that may be more relevant to (or likely to be clicked on by) the user. In addition, certain data may be anonymized in one or more ways before it is stored or used, so that personally identifiable information is removed when generating monetizable parameters (e.g., monetizable demographic parameters). For example, a user's identity may be anonymized so that no personally identifiable information can be determined for the user, or a user's geographic location may be generalized where location information is obtained (such as to a city, ZIP code, or state level), so that a particular location of a user cannot be determined. Thus, the user may have control over how information is collected (and/or used) about him or her.

While this specification contains many specific implementation details, these should not be construed as limitations on the scope of any inventions or of what may be claimed, but rather as descriptions of features specific to particular implementations of particular inventions. Certain features that are described in this specification in the context of separate implementations can also be implemented in combination in a single implementation. Conversely, various features that are described in the context of a single implementation can also be implemented in multiple implementations separately or in any suitable subcombination. Moreover, although features may be described above as acting in certain combinations and even initially claimed as such, one or more features from a claimed combination can in some cases be excised from the combination, and the claimed combination may be directed to a subcombination or variation of a subcombination.

Similarly, while operations are depicted in the drawings in a particular order, this should not be understood as requiring that such operations be performed in the particular order shown or in sequential order, or that all illustrated operations be performed, to achieve desirable results. In certain circumstances, multitasking and parallel processing may be advantageous. Moreover, the separation of various system components in the implementations described above should not be understood as requiring such separation in all implementations, and it should be understood that the described program components and systems can generally be integrated together in a single software product or packaged into multiple software products.

A number of implementations have been described. Nevertheless, it will be understood that various modifications may be made without departing from the spirit and scope of the disclosure. For example, various forms of the flows shown above may be used, with steps re-ordered, added, or removed. Accordingly, other implementations may fall within the scope of the following claims.

1. A computer-implemented method comprising:
accessing, by one or more processors, data for a plurality of health-related parameters of an individual;
determining, by the one or more processors, a corresponding individual age-adjustment factor based on data for one or more of the plurality of health-related parameters, wherein the age-adjustment factor computed for a given health-related parameter represents an estimated gain or loss in age as indicated by the data for the given health-related parameter;
combining, by the one or more processors, two or more of the individual age-adjustment factors to provide a combined age-adjustment factor; and
determining, by the one or more processors, a physiological age of the individual based on the combined age-adjustment factor and a chronological age of the individual.

2. The method of claim 1, wherein the data for the plurality of health-related parameters includes one or more of: data on levels of one or more biomarkers, data on levels of one or more physiological markers, and data on one or more lifestyle-related parameters.

3. The method of claim 2, wherein the biomarkers include one or more of glucose, testosterone, vitamin D, C-reactive protein (CRP), and alanine aminotransferase (ALT).

4. The method of claim 2, wherein the physiological markers include heart rate variability (HRV), pulse pressure, and body mass index (BMI).

5. The method of claim 2, wherein the data on the one or more lifestyle-related parameters includes one or more of: data on smoking habits and data on activity level.

6. The method of claim 1, wherein determining an individual age-adjustment factor for a particular health related parameter comprises:
determining an estimated age of the individual based on the data for the particular health-related parameter; and
determining the individual age adjustment factor as a difference between the chronological age and the estimated age.

7. The method of claim 6, wherein the estimated age is determined using an equation that relates the particular health parameter to the estimated age.

8. The method of claim 1, wherein determining an individual age-adjustment factor for a particular health related parameter comprises:
estimating a projected lifespan for the individual based on the chronological age of the individual and the data for the particular health-related parameter;
determining a standard expected lifespan for the individual based on accessing life-expectancy data for a population of which the individual is a part; and
determining the individual age adjustment factor as a difference between the projected lifespan and the standard expected lifespan.

9. The method of claim 8, wherein the projected lifespan is estimated by accessing a look-up table that stores projected lifespan values as a function of tuples comprising the chronological age of the individual and the data for the particular health-related parameter.

10. The method of claim 8, wherein the standard expected lifespan is determined by accessing life-expectancy data in an actuarial life table.

11. The method of claim 1, wherein the individual age-adjustment factor determined for a health-related parameter is capped or scaled such that the individual age-adjustment factor is within a predetermined range.

12. The method of claim 1, wherein determining the age-adjustment factor comprises:
receiving user-input in response to one or more questions presented to the individual; and
determining the individual age adjustment factor by accessing a look-up table based on the user-input.

13. The method of claim 1, wherein the health related parameters are obtained using a wearable device worn by the individual.

14. The method of claim 1, wherein the health related parameters comprise one or more genetic markers, or one or more microbiome markers.

15.-28. (canceled)

29. A system comprising:
a memory device; and
an age analysis engine comprising a processor, the age analysis engine configured to:
access data for a plurality of health-related parameters of an individual;
determine a corresponding individual age-adjustment factor based on data for one or more of the plurality of health-related parameters, wherein the age-adjustment factor computed for a given health-related parameter represents an estimated gain or loss in age as indicated by the data for the given health-related parameter;
combine two or more of the individual age-adjustment factors to provide a combined age-adjustment factor; and
determine a physiological age of the individual based on the combined age-adjustment factor and a chronological age of the individual.

30. The system of claim 29, wherein the data for the plurality of health-related parameters includes one or more of: data on levels of one or more biomarkers, data on levels of one or more physiological markers, and data on one or more lifestyle-related parameters.

31. The system of claim 30, wherein the biomarkers include one or more of glucose, testosterone, vitamin D, C-reactive protein (CRP), and alanine aminotransferase (ALT).

32. The system of claim 30, wherein the physiological markers include heart rate variability (HRV), pulse pressure, and body mass index (BMI).

33. The system of claim 30, wherein the data on the one or more lifestyle-related parameters includes one or more of: data on smoking habits and data on activity level.

34. The system of claim 29, wherein the age analysis engine is con figured to determine an individual age-adjustment factor for a particular health related parameter by:
determining an estimated age of the individual based on the data for the particular health-related parameter; and
determining the individual age adjustment factor as a difference between the chronological age and the estimated age.

35. The system of claim 34, wherein the estimated age is determined using an equation that relates the particular health parameter to the estimated age.

36. The system of claim 29, wherein the age analysis engine is configured to determine an individual age-adjustment factor for a particular health related parameter by:
estimating a projected lifespan for the individual based on the chronological age of the individual and the data for the particular health-related parameter;
determining a standard expected lifespan for the individual based on accessing life-expectancy data for a population of which the individual is a part; and
determining the individual age adjustment factor as a difference between the projected lifespan and the standard expected lifespan.

37. The system of claim 36, wherein the projected lifespan is estimated by accessing a look-up table that stores projected lifespan values as a function of tuples comprising the chronological age of the individual and the data for the particular health-related parameter.

38. The system of claim 36, wherein the standard expected lifespan is determined by accessing life-expectancy data in an actuarial life table.

39. The system of claim 29, wherein the individual age-adjustment factor determined for a health-related parameter is capped or scaled such that the individual age-adjustment factor is within a predetermined range.

40. The system of claim 29, wherein the age analysis engine is configured to determine the age-adjustment factor by:
receiving user-input in response to one or more questions presented to the individual; and
determining the individual age adjustment factor by accessing a look-up table based on the user-input.

41. The system of claim 29, wherein the health related parameters are obtained using a wearable device worn by the individual.

42. The system of claim 29, wherein the health related parameters comprise one or more genetic markers, or one or more microbiome markers.

43. A non-transitory computer readable storage device having encoded thereon computer readable instructions, which when executed, cause one or more processors to perform operations comprising:
accessing, by one or more processors, data for a plurality of health-related parameters of an individual;
determining, by the one or more processors, a corresponding individual age-adjustment factor based on data for one or more of the plurality of health-related parameters, wherein the age-adjustment factor computed for a given health-related parameter represents an estimated gain or loss in age as indicated by the data for the given health-related parameter;
combining, by the one or more processors, two or more of the individual age-adjustment factors to provide a combined age-adjustment factor; and
determining, by the one or more processors, a physiological age of the individual based on the combined age-adjustment factor and a chronological age of the individual.

44. The computer readable storage device of claim 43, wherein the data for the plurality of health-related parameters includes one or more of: data on levels of one or more biomarkers, data on levels of one or more physiological markers, and data on one or more lifestyle-related parameters.

45. The computer readable storage device of claim 44, wherein the biomarkers include one or more of glucose, testosterone, vitamin D, C-reactive protein (CRP), and alanine aminotransferase (ALT).

46. The computer readable storage device of claim 44, wherein the physiological markers include heart rate variability (HRV), pulse pressure, and body mass index (BMI).

47. The computer readable storage device of claim 44, wherein the data on the one or more lifestyle-related parameters includes one or more of: data on smoking habits and data on activity level.

48. The computer readable storage device of claim 43, wherein determining an individual age-adjustment factor for a particular health related parameter comprises:
determining an estimated age of the individual based on the data for the particular health-related parameter; and
determining the individual age adjustment factor as a difference between the chronological age and the estimated age.

49. The computer readable storage device of claim 48, wherein the estimated age is determined using an equation that relates the particular health parameter to the estimated age.

50. The computer readable storage device of claim 43, wherein determining an individual age-adjustment factor for a particular health related parameter comprises:
estimating a projected lifespan for the individual based on the chronological age of the individual and the data for the particular health-related parameter;
determining a standard expected lifespan for the individual based on accessing life-expectancy data for a population of which the individual is a part; and
determining the individual age adjustment factor as a difference between the projected lifespan and the standard expected lifespan.

51. The computer readable storage device of claim 50, wherein the projected lifespan is estimated by accessing a look-up table that stores projected lifespan values as a function of tuples comprising the chronological age of the individual and the data for the particular health-related parameter.

52. The computer readable storage device of claim 50, wherein the standard expected lifespan is determined by accessing life-expectancy data in an actuarial life table.

53. The computer readable storage device of claim 43, wherein the individual age-adjustment factor determined for a health-related parameter is capped or scaled such that the individual age-adjustment factor is within a predetermined range.

54. The computer readable storage device of claim 43, wherein determining the age-adjustment factor comprises:
receiving user-input in response to one or more questions presented to the individual; and
determining the individual age adjustment factor by accessing a look-up table based on the user-input.

55. The computer readable storage device of claim 43, wherein the health related parameters are obtained using a wearable device worn by the individual.

56. The computer readable storage device of claim 43, wherein the health related parameters comprise one or more genetic markers, or one or more microbiome markers.
